SEQUENCE LISTING (14 sequences)

SEQ ID NO: 1 - protein, 473 aa, Homo sapiens
MLKVTVPSCSASSCSSVTASAAPGTASLVPDYWIDGSNRDALSDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVRKLIVLDPKKRLTTFQALQHPWVTGKAANFVHMDTAQKKLQEFNARRKLKAAVKAVVASSRLGSASSSHGSIQESHKASRDPSPIQDGNEDMKAIPEGEKIQGDGAQAAVKGAQAELMKVQALEKVKGADINAEEAPKMVPKAVEDGIKVADLELEEGLAEEKLKTVEEAAAPREGQGSSAVGFEVPQQDVILPEY

SEQ ID NO: 2 - protein, 469 aa, Mus musculus
MLKVTVPSCPSSPCSSVTASTENLVPDYWIDGSNRDPLGDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVKKLIVLDPKKRLTTFQALQHPWVTGKAANFVHMDTAQKKLQEFNARRKLKAAVKAVVASSRLGSASSSHTSIQENHKASSDPPSTQDAKDSTDLLGKKMQEEDQEEDQVEAEASADEMRKLQSEEVEKDAGVKEEETSSMVPQDPEDELETDDPEMKRDSEEKLKSVEEEMDPMTEEEAPDAGLGVPQQDAIQPEY

SEQ ID NO: 3 - DNA, 2168 bp, Homo sapiens
ctctctcgctcctgcgttcgcaggcggcggctggcggccggcttctcgctcgggcagcggcggcggcggcggcggcggcttccggagtcccgctgcgaagatgctcaaagtcacggtgccctcctgctccgcctcgtcctgctcttcggtcaccgccagtgcggccccggggaccgcgagcctcgtcccggattactggatcgacggctccaacagggatgcgctgagcgatttcttcgaggtggagtcggagctgggacggggtgctacatccattgtgtacagatgcaaacagaaggggacccagaagccttatgctctcaaagtgttaaagaaaacagtggacaaaaaaatcgtaagaactgagataggagttcttcttcgcctctcacatccaaacattataaaacttaaagagatatttgaaacccctacagaaatcagtctggtcctagaactcgtcacaggaggagaactgtttgataggattgtggaaaagggatattacagtgagcgagatgctgcagatgccgttaaacaaatcctggaggcagttgcttatctacatgaaaatgggattgtccatcgtgatctcaaaccagagaatcttctttatgcaactccagccccagatgcaccactcaaaatcgctgattttggactctctaaaattgtggaacatcaagtgctcatgaagacagtatgtggaaccccagggtactgcgcacctgaaattcttagaggttgtgcctatggacctgaggtggacatgtggtctgtaggaataatcacctacatcttactttgtggatttgaaccattctatgatgaaagaggcgatcagttcatgttcaggagaattctgaattgtgaatattactttatctccccctggtgggatgaagtatctctaaatgccaaggacttggtcagaaaattaattgttttggatccaaagaaacggctgactacatttcaagctctccagcatccgtgggtcacaggtaaagcagccaattttgtacacatggataccgctcaaaagaagctccaagaattcaatgcccggcgtaagcttaaggcagcggtgaaggctgtggtggcctcttcgcgcctgggaagtgccagcagcagccatggcagcatccaggagagccacaaggctagccgagacccttctccaatccaagatggcaacgaggacatgaaagctattccagaaggagagaaaattcaaggcgatggggcccaagccgcagttaagggggcacaggctgagctgatgaaggtgcaagccttagagaaagttaaaggtgcagatataaatgctgaagaggcccccaaaatggtgcccaaggcagtggaggatgggataaaggtggctgacctggaactagaggagggcctagcagaggagaagctgaagactgtggaggaggcagcagctcccagagaagggcaaggaagctctgctgtgggttttgaagttccacagcaagatgtgatcctgccagagtactaaacagcttccttcagatctggaagccaaacaccggcattttatgtactttgtccttcagcaagaaaggtgtggaagcatgatatgtactatagtgattctgtttttgaggtgcaaaaaacatacatatataccagttggtaattctaacttcaatgcatgtgactgctttatgaaaataatagtgtcttctatggcatgtaatggatacctaataccgatgagttaaatcttgcaagttaacacaacgtaacacttaaaagcatacattttcagcaaccagtggcacatatttgaagtgaatagtagcaaattgtttttgctttgaaaatctagccatcctacatcctttggatttcttcacaaggcagtaattcctttgaactactgcttagctaatactaggtagtgctaaaagacatgttcccataacttttacaacattttactttttatcattgatgtgttcaaactgtttacaaggagatgcttatagatgatagttgtacatatgtgcaaaaaaaaatccacttgcaatggtaagaaattgaagtatccttaaaggccatgaagccatatgtccctaaaaaaaaaaaaaaaaaaaaaaaaaaaaaaaaaaaaaaaaaaaaaaaaaaaaaaaaaaaaaaaa

SEQ ID NO: 4 - DNA, 1805 bp, Mus musculus
ggttccctgcctggtctctgccggctggcggtggctccgggttcccctgcgccggtgctcgcggtctcgggcatctccgcccgccctcgctcccgagcgcggaggcgagccgcagcccagcgcggcggtgggcgtgtgcgcgctgaaggacgcctcctctcgctcccgtgttgcaggcggcggccggcggctcgagctccggcagcggcggcgtcttccggagtcccgcggcgaagatgctcaaagtcacggtgccctcctgtccctcctcgccctgctcctcggtcaccgccagtactgagaacctcgtcccggattactggatcgacggctctaaccgagatcctctgggcgatttcttcgaggtggagtcagagctgggacggggtgctacatccattgtgtacagatgcaaacagaaggggacccagaagccctatgctctcaaagtgttaaagaaaacagtggacaagaagattgtgagaacagaaataggagttctcctgcgtctctcacacccgaacatcataaaactcaaggaaatattcgaaacccccacagaaatcagcctggtccttgagctggtcacaggaggagaactgtttgacaggattgtggagaagggatactacagtgagcgcgatgcggctgacgcggtgaagcagatcctggaggccgttgcttacctgcatgaaaatgggattgtccatcgtgacctcaaaccagagaatcttctttatgcaactccagcccctgatgcacccctcaaaattgctgattttggactttcaaaaattgtggaacatcaagtgctcatgaagacagtgtgtggaaccccggggtactgcgcacctgagattctccgaggctgtgcctacggacctgaggtggacatgtggtctgtaggaataatcacctacatcctactttgtggatttgaaccattctatgacgagcgaggtgatcagttcatgttcaggagaattctgaattgtgaatattactttatctccccctggtgggatgaagtgtctttaaatgccaaggacttggtcaaaaagctcattgttttggatcccaagaaacggctgactacatttcaagccctccaacacccatgggtcacaggtaaagcggccaactttgttcacatggacactgctcagaagaaacttcaagaattcaatgctcggcgcaagcttaaggcagcggtgaaggctgtggtggcctcttctcggctgggaagtgccagcagtagccacaccagcatccaagagaaccacaaggccagctcggatccaccttcaacccaagatgccaaggacagcacagatcttctgggaaagaaaatgcaagaggaggaccaagaggtggaccaagtggaggccgaggcttcagccgatgagatgaggaagctgcagtccgaggaggtggagaaagatgcaggtgtaaaagaggaggagacctccagtatggtgcctcaggatccagaggatgagctggaaacagatgacccagagatgaagagggattcagaggagaagctgaagagtgtggaggaagaaatggaccccatgactgaggaggaagcccctgacgcgggacttggggttccacagcaggatgcgattcagccagagtactaagctggcttcctttaggtcagggccaaccccagcattttatgcactctgtccctcagcaaggcggggaagcatgatatgcactatagtgattctgttttgaggtgcaaaaaaaaaacccatatataccagttggtaaccctaaaaaaaaaaaaaaaaaaa

SEQ ID NO: 5 - DNA, 22 bp, Artificial Sequence
  Synthetic primer construct
cagcagcgatcatccctcaaag

SEQ ID NO: 6 - DNA, 21 bp, Artificial Sequence
  Synthetic primer construct
caggaccaggatctcttgctg

SEQ ID NO: 7 - DNA, 25 bp, Artificial Sequence
  Synthetic primer construct
ggcaggtctactttggagtcattgc

SEQ ID NO: 8 - DNA, 25 bp, Artificial Sequence
  Synthetic primer construct
acattcgaggctccagtgaattcgg

SEQ ID NO: 9 - DNA, 22 bp, Artificial Sequence
  Synthetic primer construct
cacggcacagtcattgaaagcc

SEQ ID NO: 10 - DNA, 22 bp, Artificial Sequence
  Synthetic primer construct
cttattgggacaatctcttccc

SEQ ID NO: 11 - DNA, 17 bp, Artificial Sequence
  Synthetic primer construct
cgaaatccaggatgccc

SEQ ID NO: 12 - DNA, 20 bp, Artificial Sequence
  Synthetic primer construct
gacaccagtatcttctccag

SEQ ID NO: 13 - DNA, 20 bp, Artificial Sequence
  Synthetic primer construct
actcaacacgggaaacctca

SEQ ID NO: 14 - DNA, 20 bp, Artificial Sequence
  Synthetic primer construct
aaccagacaaatcgctccac

(19) United States
(12) Patent Application Publication
(10) Pub. No.: US 2014/0161769 A1
(43) Pub. Date: Jun. 12, 2014

(54) METHODS FOR TREATING INFLAMMATORY AUTOIMMUNE DISORDERS

(71) Applicant: Beth Israel Deaconess Medical Center, Inc., Boston, MA (US)

(72) Inventors: George C. Tsokos, Boston, MA (US); Yuang-Taung Juang, Newton, MA (US); Chun-Shin Hahn, Cambridge, MA (US)

(73) Assignee: Beth Israel Deaconess Medical Center, Inc., Boston, MA (US)

(21) Appl. No.: 13/936,792

(22) Filed: Jul. 8, 2013

Related U.S. Application Data
(63) Continuation of application No. 13/698,879, now abandoned.
(60) Provisional application No. 61/346,212, filed on May 19, 2010.

(51) Int. Cl. A61K 31/18; A61K 31/713; A61K 45/06; C12N 15/113

(52) U.S. Cl. CPC A61K 31/18; C12N 15/1137; A61K 31/713; A61K 45/06
USPC 424/85.4; 514/44 A; 514/604; 514/171; 424/133.1; 514/20.5; 424/142.1; 424/134.1; 514/263.2; 435/7.4

(57) ABSTRACT

The present invention features a method of treating or reducing the likelihood of an inflammatory autoimmune disorder (e.g., systemic lupus erythematosus (SLE)) or a kidney disorder (e.g., glomerulonephritis) in a subject by administering an inhibitor of calcium/calmodulin-dependent protein kinase type IV (CaMKIV). The invention additionally features methods of diagnosing a subject with SLE or a kidney disorder by determining the level or biological activity of a CaMKIV nucleic acid or polypeptide in a sample from a subject. Also included in the invention are methods for identifying compounds that inhibit CaMKIV for the treatment of an inflammatory autoimmune disorder (e.g., SLE) or a kidney disorder (e.g., glomerulonephritis).

### CROSS-REFERENCE TO RELATED APPLICATIONS

This application is a continuation of U.S. patent application Ser. No. 13/698,879, filed Nov. 19, 2012, which claims priority under 35 U.S.C. §371 from International application no. PCT/US2011/037181, filed on May 19, 2011, which claims priority under 35 U.S.C. §119 from U.S. provisional application No. 61/346,212, filed on May 19, 2010, each of which are herein incorporated by reference.

### STATEMENT AS TO FEDERALLY SPONSORED RESEARCH

This invention was made with government support under grant number PHS NIH NIAID RO1 AI 49954 awarded by the National Institutes of Health. The government has certain rights in the invention.

### BACKGROUND OF THE INVENTION

Systemic lupus erythematosus (SLE) is a chronic inflammatory autoimmune disorder that most commonly affects the skin, joints, kidneys, heart, lungs, liver, nervous system, blood vessels, and brain. As occurs in other autoimmune diseases, the immune system attacks the body's cells and tissue, typically resulting in inflammation and tissue damage. The course of the disease is unpredictable, with periods of illness alternating with periods of remission. There is no cure for SLE, and treatment is aimed at controlling the symptoms of SLE, often with the use of indiscriminate immunosuppressive agents. Furthermore, autoimmune disorders generally implicate disorders in other organs, such as in the kidney. Accordingly, improvements are needed for the treatment of SLE, other inflammatory autoimmune disorders, and associated disorders, such as kidney disorders.

### SUMMARY OF THE INVENTION

In general, the present invention features a method of treating or reducing the likelihood of an inflammatory autoimmune disorder (e.g., SLE) or a kidney disorder (e.g., glomerulonephritis) in a subject by providing an inhibitor of calcium/calmodulin-dependent protein kinase type IV (CaMKIV). The invention additionally features methods of diagnosing a subject with an inflammatory autoimmune disorder (e.g., SLE) or a kidney disorder (e.g., glomerulonephritis) by determining the level or biological activity of a CaMKIV nucleic acid or polypeptide in a sample from a subject. Also included in the present invention are methods for identifying compounds that inhibit CaMKIV for the treatment of an inflammatory autoimmune disorder (e.g., SLE) or a kidney disorder (e.g., glomerulonephritis).

In a first aspect, the invention features a method of treating an inflammatory autoimmune disorder or a kidney disorder or reducing the likelihood of developing an inflammatory autoimmune disorder or a kidney disorder in a subject by providing to a subject an inhibitor of CaMKIV, wherein the inhibitor is provided in an amount and for a duration that together are sufficient to treat an inflammatory autoimmune disorder or a kidney disorder or reduce the likelihood of developing an inflammatory autoimmune disorder or a kidney disorder in a subject. The inhibitor may reduce or inhibit the biological activity (e.g., kinase activity) or expression level of a CaMKIV protein or nucleic acid molecule. Exemplary inhibitors include small molecules (e.g., KN-93) and nucleic acid molecules (e.g., siRNA). In certain embodiments, the methods of the invention may further include providing to a subject an additional therapeutic agent (e.g., adalimumab, azathioprine, chloroquine, hydroxychloroquine, ciclosporin, D-penicillamine, etanercept, golimumab, auranofin, infliximab, leflunomide, methotrexate, minocycline, rituximab, sulfasalazine, plaquenil, cyclophosphamide, tacrolimus, sirolimus, dehydroepiandrosterone, an opiate, an interferon, a corticosteroid, or a nonsteroidal anti-inflammatory drug).

In another aspect, the invention features a method of diagnosing a subject as having an inflammatory autoimmune disorder or a kidney disorder by determining the level or biological activity of a CaMKIV nucleic acid or polypeptide, or fragments thereof, in a sample from a subject and comparing it to a reference, wherein an increase in the level or biological activity of CaMKIV nucleic acid or polypeptide, or fragments thereof, compared to a reference is a diagnostic indicator of an inflammatory autoimmune disorder or a kidney disorder in a subject. The sample may be a bodily fluid (e.g., urine, blood, serum, plasma, or cerebrospinal fluid), cell, or tissue sample from a subject in which CaMKIV nucleic acid or polypeptide is normally detectable.

The invention also features a method of identifying a candidate compound useful for treating an inflammatory autoimmune disorder or a kidney disorder in a subject by contacting a CaMKIV polypeptide, or a fragment thereof, with a compound and measuring the biological activity (e.g., kinase activity) of a CaMKIV polypeptide, or fragment thereof, wherein a decrease in CaMKIV biological activity in the presence of a compound relative to CaMKIV biological activity in the absence of a compound identifies a compound as a candidate compound for treating an inflammatory autoimmune disorder or a kidney disorder in a subject.

The invention additionally features a method of identifying a candidate compound useful for treating an inflammatory autoimmune disorder or a kidney disorder in a subject by contacting a cell or cell extract that includes a polynucleotide encoding CaMKIV with a compound and measuring the level of CaMKIV expression in a cell or cell extract, wherein a decreased level of CaMKIV expression in the presence of a compound relative to the level in the absence of a compound identifies a compound as a candidate compound for treating an inflammatory autoimmune disorder or a kidney disorder in a subject.

In one embodiment in any of the above methods, the inflammatory autoimmune disorder is systemic lupus erythematosus (SLE). In another embodiment, the inflammatory autoimmune disorder is Hashimoto's thyroiditis, pernicious anemia, Addison's disease, type I diabetes, rheumatoid arthritis, dermatomyositis, Sjögren's syndrome, lupus erythematosus, multiple sclerosis, myasthenia gravis, reactive arthritis, Grave's disease, or celiac disease. In another embodiment in any of the above methods, the inflammatory autoimmune disorder is associated with a kidney disorder (e.g., lupus nephritis or glomerulonephritis). In some embodiments, the inflammatory immune disorder is SLE associated with lupus nephritis or glomerulonephritis.

In one embodiment in any of the above methods, the kidney disorder is glomerulonephritis, IgA nephropathy, lupus nephritis, diabetic nephropathy, or glomerulosclerosis. In particular embodiments, the kidney disorder is glomerulonephritis (e.g., membranoproliferative glomerulonephritis or post-streptococcal glomerulonephritis).

By “an amount sufficient” is meant the amount of a compound or therapeutic agent, alone or in combination with another compound, therapeutic agent, or therapeutic regimen, required to treat or ameliorate a disorder, such as an inflammatory autoimmune disorder (e.g., SLE) or a kidney disorder (e.g., glomerulonephritis), in a clinically relevant manner. A sufficient amount of a compound or therapeutic agent used to practice the present invention for therapeutic treatment of, e.g., an inflammatory autoimmune disorder (e.g., SLE) or a kidney disorder (e.g., glomerulonephritis) varies depending upon the manner of administration, age, and general health of the subject. Ultimately, the medical practitioner prescribing such treatment will decide the appropriate amount and dosage regimen. Additionally, a sufficient amount may be an amount of compound in a combination of therapeutic agents that is safe and efficacious in the treatment of a subject having a disorder over each agent alone.

By “biological sample” or “sample” is meant solid and fluid samples. Biological samples may include cells, protein or membrane extracts of cells, blood or biological fluids including, e.g., ascites fluid or brain fluid (e.g., cerebrospinal fluid (CSF)). Examples of solid biological samples include samples taken from the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, the thymus, and feces. Examples of biological fluid samples include blood, serum, CSF, semen, prostate fluid, seminal fluid, urine, saliva, tears, sputum, mucus, bone marrow, and lymph samples. Samples may be obtained by standard methods including, e.g., venous puncture and surgical biopsy.

By “calcium/calmodulin-dependent protein kinase type IV” or “CaMKIV” is meant a polypeptide, or a nucleic acid sequence that encodes it, or fragments or derivatives thereof, that is substantially identical or homologous to or encodes any protein substantially identical to the amino acid set forth in GenBank Accession Numbers NP_{—}001735 (human; SEQ ID NO: 1) and/or NP_{—}033923 (mouse; SEQ ID NO: 2). (See, e.g., FIG. 11.) CaMKIV can also include fragments, derivatives, homologs, orthologs, or analogs of CaMKIV that retain at least 10%, 15%, 20%, 25%, 30%, 40%, 50%, 60%, 70%, 80%, 90%, 95%, 96%, 97%, 98%, 99%, or more CaMKIV biological activity. The CaMKIV polypeptides may be isolated from a variety of sources, such as from mammalian tissue, plasma, or cells, or from another source, or prepared by recombinant or synthetic methods. The term “CaMKIV” also encompasses modifications to the polypeptide, fragments, derivatives, analogs, and variants of the CaMKIV polypeptide having CaMKIV biological activity.

By “CaMKIV biological activity” is meant any one or more of the following activities: kinase activity, promotion of the expression of IL-17, promotion of the expression of B cell CD86, induction of the expression of IL-21, production of anti-dsDNA antibodies, promotion of the expression of a cytokine (e.g., IFN-γ, IL-1β, IL-6, and/or TNF-α), promotion of the expression of CD69, or promotion of the expression of a cyclin associated with cell proliferation (e.g., CDK2 or cyclin DD.

By “CaMKIV inhibitor” is meant any compound which inhibits the biological activity of CaMKIV or expression of CaMKIV. A CaMKIV inhibitor may inhibit the kinase activity of CaMKIV or any other biological activity of CaMKIV described herein. Compounds may be identified as CaMKIV inhibitors by evaluating the compounds in assays known to one of skill in the art and described herein. Known inhibitors of CaMKIV include, for example, ST0609 (7-oxo-7H-benzimidazo[2,1-a]benz[de]isoquinoline-3-carboxylic acid acetate), KN-93 (N-[2-[[[3-(4-chlorophenyl)-2-propenyl]methylamino]methyl]phenyl]-N-(2-hydroxyethyl)-4-methoxybenzenesulphonamide) (see, e.g., Sumi et al., Biochem. Biophys. Res. Comm. 181: 968-975, 1991), KN-62 (1-[N,O-bis(5-isoquinolinesulfonyl)-N-methyl-L-tyrosyl]-4-phenylpiperazine), and K252A (see, e.g., Yoshida et al., Biochim. Biophys. Acta 1497: 155-167, 2000).

By “candidate compound” or “compound” is meant a chemical, e.g., naturally-occurring or artificially-derived. Candidate compounds may include, for example, peptides, polypeptides, synthetic organic molecules, naturally-occurring organic molecules, nucleic acid molecules (e.g., siRNA), peptide nucleic acid molecules, and components and derivatives thereof.

Compounds useful in the invention include those described herein in any of their pharmaceutically acceptable forms, including isomers, such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

By “fragment” is meant a portion of a nucleic acid or polypeptide that contains at least, e.g., 10%, 20%, 30%, 40%, 50%, 60%, 70%, 80%, 90%, 95%, or more of the entire length of the nucleic acid or polypeptide (e.g., CaMKIV nucleic acid or polypeptide). A nucleic acid fragment may contain, e.g., 10, 20, 30, 40, 50, 60, 70, 80, 90, or 100, 200, 300, 400, 500, 600, 700, 800, 900, 1000, 1100, 1200, 1300, 1400, 1500, 2000, 2500, 3000, 4000, 4500, or 5000 nucleotides or more nucleotides, up to the full length of the nucleic acid. A polypeptide fragment may contain, e.g., 10, 20, 30, 40, 50, 60, 70, 80, 90, 100, 150, 200, 250, 300, 350, 400, 450, or 500 amino acids or more amino acids, up to the full length of the polypeptide. Fragments useful in the therapeutic methods of the invention include, e.g., fragments that retain biological activity. Fragments can be modified as described herein and as known in the art.

By “inflammatory autoimmune disorder” is meant a condition that occurs when the immune system mistakenly attacks and destroys healthy cells and tissue, resulting in inflammation and tissue damage. Exemplary inflammatory autoimmune disorders include Hashimoto's thyroiditis, pernicious anemia, Addison's disease, type I diabetes, rheumatoid arthritis, SLE, dermatomyositis, Sjögren's syndrome, lupus erythematosus (e.g., discoid lupus erythematosus, drug-induced lupus erythematosus, and neonatal lupus erythematosus), multiple sclerosis, myasthenia gravis, reactive arthritis, Grave's disease, and celiac disease (e.g., gluten sensitive enteropathy). Symptoms of inflammatory autoimmune disorders include, e.g., arthritis, fatigue, fever, general discomfort or malaise, joint pain and swelling, muscle aches, nausea and vomiting, pleural effusions, pleurisy, psychosis, seizures, sensitivity to sunlight, skin rashes (e.g., facial “butterfly” rashes), swollen glands, abdominal pain, blood disorders (e.g., blood clots), blood in the urine, coughing up blood, fingers that change color upon pressure or in cold temperatures, hair loss, mouth sores, nosebleeds, numbness and tingling, red spots on skin, patchy skin coloring, difficulty swallowing, or visual disturbances.

By “inhibitor” is meant any compound (peptidyl or non-peptidyl), antibody, nucleic acid molecule, polypeptide, or fragments thereof that reduces or inhibits the expression levels or biological activity of a protein or nucleic acid molecule by at least 10%, 20%, 30%, 40%, 50%, 60%, 70%, 80%, 90%, 95%, 99% or more. Non-limiting examples of inhibitor compounds include dominant negative fragments or mutant polypeptides that block the biological activity of the wild-type protein; peptidyl or non-peptidyl compounds (e.g., antibodies or antigen-binding fragments thereof) that bind to a protein, for example at a functional domain or substrate binding domain; antisense nucleobase oligomers; morpholinos; double-stranded RNA for RNA interference; small molecule inhibitors; compounds that decrease the half-life of an mRNA or protein; and compounds that decrease transcription or translation of a polypeptide.

By “pharmaceutically acceptable carrier” is meant a carrier that is physiologically acceptable to the treated subject while retaining the therapeutic properties of the composition with which it is administered. One exemplary pharmaceutically acceptable carrier substance is physiological saline. Other physiologically acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

By “protein,” “polypeptide,” “polypeptide fragment,” or “peptide” is meant any chain of two or more amino acid residues, regardless of post-translational modification (e.g., glycosylation or phosphorylation), constituting all or part of a naturally occurring polypeptide or peptide or constituting a non-naturally occurring polypeptide or peptide. A polypeptide or peptide may be said to be “isolated” or “substantially pure” when physical, mechanical, or chemical methods have been employed to remove the polypeptide from cellular constituents. An “isolated polypeptide,” “substantially pure polypeptide,” or “substantially pure and isolated polypeptide” is typically considered removed from cellular constituents and substantially pure when it is at least 60% by weight free from the proteins and naturally occurring organic molecules with which it is naturally associated. The polypeptide may be at least 75%, 80%, 85%, 90%, 95%, or 99% by weight pure. A substantially pure polypeptide may be obtained by standard techniques, for example, by extraction from a natural source (e.g., cell lines or biological fluids), by expression of a recombinant nucleic acid encoding the polypeptide, or by chemically synthesizing the polypeptide. Purity can be measured by any appropriate method, e.g., by column chromatography, polyacrylamide gel electrophoresis, or HPLC analysis. Alternatively, a polypeptide is considered isolated if it has been altered by human intervention, placed in a location that is not its natural site, or if it is introduced into one or more cells.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the peptides or polypeptides of the invention. The mimetic can be either entirely composed of synthetic, non-natural analogs of amino acids or may be a chimeric molecule of natural amino acids and non-natural analogs of amino acids. The mimetic can also incorporate any amount of conservative substitutions, as long as such substitutions do not substantially alter the mimetic's structure or activity.

By “reduce or inhibit” is meant the ability to cause an overall decrease of 20% or greater, of 50% or greater, or of 75%, 80%, 85%, 90%, 95%, or greater. For therapeutic applications, to “reduce or inhibit” can refer to the symptoms of the disorder being treated or the presence or extent of a disorder being treated. For diagnostic or monitoring applications, to “reduce or inhibit” can refer to a decrease in the level of protein or nucleic acid detected by the diagnostic or monitoring assays.

By “reducing the likelihood of” is meant reducing the severity, the frequency, and/or the duration of a disorder (e.g., an inflammatory autoimmune disorder (e.g., SLE) or a kidney disorder (e.g., glomerulonephritis)) or symptoms thereof. Reducing the likelihood of an inflammatory autoimmune disorder or a kidney disorder is synonymous with prophylaxis or the chronic treatment of an inflammatory autoimmune disorder or a kidney disorder.

By “reference” is meant any sample, standard, or level that is used for comparison purposes. A “normal reference sample” can be a prior sample taken from the same subject prior to the onset of a disorder (e.g., an inflammatory autoimmune disorder (e.g., SLE) or a kidney disorder (e.g., glomerulonephritis)), a sample from a subject not having the disorder, a subject that has been successfully treated for the disorder, or a sample of a purified reference polypeptide at a known normal concentration. By “reference standard or level” is meant a value or number derived from a reference sample. A normal reference standard or level can be a value or number derived from a normal subject that is matched to a sample of a subject by at least one of the following criteria: age, weight, disease stage, and overall health. In one example, a normal reference level of, for example, a polypeptide indicative of a disorder is less than 5 ng/ml in a serum sample, less than 4 ng/ml, less than 3 ng/ml, less than 2 ng/ml, or less than 1 ng/ml in a serum sample. A “positive reference” sample, standard, or value is a sample, standard, value, or number derived from a subject that is known to have a disorder (e.g., an inflammatory autoimmune disorder or a kidney disorder) that is matched to a sample of a subject by at least one of the following criteria: age, weight, disease stage, and overall health. For example, a positive reference value for, e.g., a polypeptide indicative of a disorder, is greater than 5 ng/ml serum, greater than 10 ng/ml serum, greater than 20 ng/ml, greater than 30 ng/ml, greater than 40 ng/ml, or greater than 50 ng/ml serum.

By “subject” is meant a mammal, including, but not limited to, a human or non-human mammal, such as a bovine, equine, canine, ovine, or feline.

By “therapeutic agent” is meant any agent that produces a healing, curative, stabilizing, or ameliorative effect.

By “therapeutic amount” is meant an amount that, when administered to a subject suffering from a disorder (e.g., an inflammatory autoimmune disorder (e.g., SLE) or a kidney disorder (e.g., glomerulonephritis)), is sufficient to cause a qualitative or quantitative reduction in the symptoms associated with the disorder.

By “treating” or “ameliorating” is meant administering a composition (e.g., a pharmaceutical composition) for therapeutic purposes or administering treatment to a subject already suffering from a disorder to improve the subject's condition. By “treating a disorder” or “ameliorating a disorder” is meant that the disorder and/or the symptoms associated with the disorder are, e.g., alleviated, reduced, cured, or placed in a state of remission.

Other features and advantages of the invention will be apparent from the detailed description and from the claims.

### BRIEF DESCRIPTION OF THE DRAWINGS

FIG. 1 shows the genotyping of MRL/lpr and MRL/lprCaMKIV−/− and CaMKIV expression. FIG. 1A is a genotyping PCR for wild-type Fas (179 bp) and lpr mutant (217 bp) alleles in C57B6/L, MRL/lpr, and MRL/lprCaMKIV−/− mice. FIG. 1B is a genotyping PCR for WT (150 bp) and CaMKIV-null (280 bp) alleles in MRL/lpr and MRL/lprCaMKIV−/− mice (Neg: negative control). FIG. 1C is an immunoblot for CaMKIV expression in spleen and lymph node extracts, demonstrating increased CaMKIV expression in MRL/lpr mice compared to C57BL/6 and MRL/MPJ mice.

FIG. 2 shows that CaMKIV deficiency suppresses disease expression in MRL/lpr mice. FIG. 2A is a representative spleen (left panel) and lymph node (right panel) from 24-week-old MRL/lpr and MRL/lprCaMKIV−/− mice. FIG. 2B is a set of graphs showing spleen (left panel) and lymph node (right panel) weight from 16- and 24-week-old mice, presented as organ/body weight ratio (P≦0.0001; n≧9 mice per group). FIG. 2C is a series of pictures showing facial and body skin lesions in 24-week-old MRL/lpr and MRL/lprCaMKIV−/− mice. FIG. 2D is a set of representative skin sections stained with Hematoxylin-Eosin (HE) (MRL/lpr: left panel; MRL/lprCaMKIV−/−: right panel). The magnification is ×10.

FIG. 3 shows that CaMKIV deficiency improves lupus kidney pathology. FIG. 3A is a series of photographs of kidney sections (HE) representative of glomerular (left), tubular (middle), and perivascular (right) lesions for 16-week-old mice. FIG. 3B is a series of bar graphs showing mean scores of glomerular injury, tubular damage, and perivascular lymphocyte infiltration from MRL/lpr and MRL/lprCaMKIV−/− mice (*P=0.01; **P=0.009; ***P≦0.001; n≧9 mice per group). A Kruskal-Wallis test was used for the statistical analyses. Bars represent mean+SD. The magnification is ×40 (glomerular), ×10 (tubular), and ×20 (perivascular).

FIG. 4 shows that CaMKIV deficiency limits renal disease. FIG. 4A is a graph showing the weekly quantification of protein and leukocytes in urine during an 18-week period starting when the mice were 6-weeks-old. The mice in each group were placed overnight in a Nalgene metabolic cage to collect urine. FIG. 4B is a graphical representation of an ELISA. Anti-dsDNA IgG antibodies were detected by ELISA (*P≦0.0001; n≧9 mice per group). FIGS. 4C and 4D are immunofluorescence images. For kidney immunofluorescence examination, cryostat-sectioned tissues were stained with FITC-conjugated antibodies against mouse IgG (FIG. 4C) and C3 (FIG. 4D). Representative images of IgG and C3 in MRL/lpr (left) and MRL/lprCaMKIV−/− (middle) mice are shown. As a negative control, sections from healthy C57BL/6 mice were included (right). The magnification is ×60.

FIG. 5 shows that CaMKIV deficiency suppresses pro-inflammatory cytokine production in MRL/lpr mice. FIG. 5A is a bar graph showing the quantification of IFN-γ (left) and TNF-α (right) levels in sera from 16- and 24-week-old mice (*P=0.009; **P=0.008; ***P=0.006; n≧9 mice per group). FIG. 5B is a gel showing mRNA levels of IFN-γ (first two lanes), TNF-α (second two lanes), and IL-17A (last two lanes) from isolated splenocytes incubated with PBS, CD3, or CD3/CD28 during a 72-hour time period. CaMKIV +/+ denotes MRL/lpr; −/− denotes MRL/lprCaMKIV−/−. 18srRNA was used as a RNA loading control. FIG. 5C is a series of bar graphs showing IFN-γ (left), TNF-α(middle), and IL-17A (right) levels in supernatants from splenocytes incubated with PBS, CD3 or CD3/CD28 during a 72-hour time period (*P=0.02; **P≦0.001; n=five 24-week-old mice per group). A Kruskal-Wallis test was used for statistical analyses. Results are expressed as mean+SD.

FIG. 6 shows that CaMKIV inhibition results in elimination of IL-17 T cells from the kidneys of MRL/lpr mice and IL-17A production in human T cells. FIG. 6A is a series of representative photographs of frozen sections of an MRL/lpr (left panels) and an MRL/lprCaMKIV−/− mouse (right panels) stained with rat anti-mouse CD3 and biotin anti-mouse IL-17A followed by Alexa Fluor (AF) 488-labeled anti-rat antibody and AF 568-labeled streptavidin. Sections were scanned in a confocal microscope. IL-17A positive T cells are indicated by a white arrow. The magnification is ×60. FIGS. 6B and 6C are RT-PCR experiments, wherein T cells from a healthy control (FIG. 6B) and a patient with SLE (FIG. 6C) were transfected with either control siRNA or CaMKIV siRNA. After 48 hours, cells were stimulated with CD3/CD28 for 5 hours, and IL-17A mRNA was quantified by RT-PCR. 18srRNA was used as a RNA loading control. One of three similar experiments is shown.

FIG. 7 shows that the treatment of MRL/lpr mice with the CaMKIV inhibitor KN-93 ameliorates lupus nephritis. FIG. 7A is a graph showing the weekly quantification of urinary protein and leukocytes of pre-disease 8-week-old MRL/lpr mice treated with either KN-93 (40 mg/kg) or PBS. The agent was administered by intraperitoneal injections three times a week, every other week, during an 8-week time period. The arrows indicate the administration of the treatment. FIG. 7B is a graph showing the weekly quantification of urinary protein and leukocytes of diseased 12-week-old MRL/lpr mice (n≧5 mice per group) treated with KN-93 or PBS. Treatment was administered three times a week during a 5-week time period. FIG. 7C is a series of representative images of glomerular, tubule-interstitial, and perivascular areas from 16-week-old MRL/lpr mice treated with PBS (upper panels) and KN-93 (stained with Periodic Acid Schiff). The photographs from the middle panels correspond to mice treated before the onset of proteinuria (8 week-old). The lower panels correspond to mice treated after the disease had been established. The magnification is ×40 (glomerular), ×10 (tubular), and ×20 (perivascular).

FIG. 8 shows decreased expression of CD86, but not CD80, in spleens of MRL/lprCaMKIV −/− mice. FIGS. 8A and 8B are immunoblots of protein extracts obtained from spleen and immunoblotted with anti-CD86 (FIG. 8A) and anti-CD80 antibodies (FIG. 8B) (representative of two experiments using three mice in each experiment). Splenocytes were isolated from MRL/lpr and MRL/lprCaMKIV−/− mice and stimulated with LPS (1 μg/ml) or PBS for 24 hours. Cumulative data for CD86 (FIG. 8C) and CD80 (FIG. 8E) from three mice are presented as mean+SD. A Kruskal-Wallis test was used for statistical analyses (P≦0.0001; n=three 24-week-old mice per group). Representative histograms comparing CD86 (FIG. 8D) and CD80 (FIG. 8F) expression in splenocytes from unstimulated cells (dotted line) and LPS stimulated cell (solid line) are shown. The shaded areas represent an isotype control.

FIG. 9 shows that CD86 expression is decreased in B cells from MRL/lprCaMKIV−/− mice. FIGS. 9A, 9B, and 9C are bar graphs showing the expression of CD86/CD19 (FIG. 9A), CD86/CD11c (FIG. 9B), and CD86/F4-80 (FIG. 9C). Splenocytes were isolated from MRL/lpr and MRL/lprCaMKIV−/− mice and stimulated with LPS (1 μg/ml) or PBS for 0 hours and 24 hours. Cumulative data of CD86 and CD19, CD11c, and F4/80 from three independent mice are presented as mean+SD. A Kruskal-Wallis test was used for statistical analyses (*P≦0.01; **P≦0.001; *** P≦0.0001; n=three 24-week-old mice per group). FIG. 9D is a series of representative histograms comparing CD19, CD11c, and F4/80 expression in splenocytes from LPS-stimulated cells (solid line) and unstimulated cells (dotted line) for 24 hours. The shaded areas represent an isotype control. One of three representative results is shown.

FIG. 10 shows that KN-93 decreases CD86 expression on splenocytes from MRL/lpr mice. Splenocytes were isolated from MRL/lpr mice and stimulated with LPS (1 μg/ml) or PBS for 24 hours. FIGS. 10A and 10C are bar graphs showing the expression of CD86 in LPS-stimulated cells (FIG. 10A) and unstimulated cells (FIG. 10C). A Kruskal-Wallis test was used for statistical analyses. Results are expressed as mean+SD CP=0.01; n=three 24-week-old mice per group). FIGS. 10B and 10D are representative histograms comparing CD86 expression on LPS-stimulated (FIG. 10B) and unstimulated splenocytes (FIG. 10D). Splenocytes were treated with KN-93 (10 μM) (right panel) or PBS (left panel) for 24 hours. Shaded areas represent isotype control staining.

FIG. 11 shows the polypeptide sequences of human CaMKIV (SEQ ID NO: 1) and murine CaMKIV (SEQ ID NO: 2).

FIG. 12 shows the nucleic acid sequence of human CaMKIV (SEQ ID NO: 3).

FIG. 13 shows the nucleic acid sequence of murine CaMKIV (SEQ ID NO: 4).

FIG. 14 shows that the treatment of MRL/lpr mice with the CaMKIV inhibitor KN-93 ameliorates lupus nephritis. FIGS. 14A-14C are graphs showing the weekly quantification of urinary protein of pre-disease 8-week-old MRL/lpr mice treated with either KN-93 (40 mg/kg) or PBS. The agent was administered by intraperitoneal injections three times a week, and the arrows indicate the administration of the treatment. Treatment groups included administration every other week during weeks 8 to 16 in group A (FIG. 14A), every week during weeks 12 to 16 in group B (FIG. 14B), and every week during weeks 15 to 18 in group C (FIG. 14C).

FIG. 15 shows that treatment with the CaMKIV inhibitor KN-93 decreases cytokine production in normal human T cells and macrophages. FIG. 15A is a bar graph showing IFN-γ levels in supernatants from human T cells incubated with PBS or CD3/28 and treated with either PBS (white bars) or with KN-93 (black bars). FIG. 15B are histograms comparing CD69 expression in human T cells incubated with PBS (left) or with CD3/28 (right). Data are shown for control (gray), treatment with PBS (dotted line), and treatment with KN-93 (solid line). FIG. 15C is a series of bar graphs showing IL-113 (left), IL-6 (middle), and TNF-α(right) levels in supernatants from macrophages incubated with PBS or LPS and treated with either PBS (white bars) or with KN-93 (black bars).

FIG. 16 shows that inhibition of CaMKIV with KN-93 inhibits mesangial cell proliferation with or without PDGF stimulation, where PDGF enhances further mesangial cell proliferation. FIG. 16A is a series of histograms in unstimulated cells (left) and cells stimulated with PDGF (right). FIG. 16B is a series of immunoblots of protein extracts obtained from murine mesangial cells and immunoblotted with anti-CDK2 or anti-cyclin D1 antibodies. After isolation from MRL/MPJ or MRL/lpr mice, these cells were untreated, treated with KN-93, treated with PDGF, or treated with both KN-93 and PDGF.

FIG. 17 shows that genetic elimination of CaMKIV inhibits mesangial cell proliferation with or without PDGF stimulation. FIG. 17A is a series of histograms in unstimulated cells (left) or cells stimulated with PDGF (right). FIG. 17B is a series of immunoblots of protein extracts obtained from murine mesangial cells and immunoblotted with anti-CDK2 or anti-cyclin D1 antibodies. After isolation from MRL/MPJ, MRL/lpr, and MRL/lprCaMKIV−/− mice, these cells were untreated, treated with KN-93, treated with PDGF, or treated with both KN-93 and PDGF. FIGS. 17C and D show CaMKIV expression in murine mesangial cells, where mRNA levels of CaMKIV (FIG. 17C) and protein levels of CaMKIV (FIG. 17D) are shown.

FIG. 18 shows that IL-6 expression is suppressed by elimination of CaMKIV. FIGS. 18A and 18B are gels showing mRNA levels of IL-6 in murine cells. 18srRNA was used as a RNA loading control. FIGS. 18C and 18D are bar graphs showing IL-6 levels in supernatants from murine cells. After isolation from MRL/MPJ (“MPJ”), MRL/lpr (“lpr”), and MRL/lprCaMKIV−/− (“CaMKIV−/−”), these murine cells were unstimulated, treated with KN-93, treated with PDGF, or treated with both KN-93 and PDGF.

### DETAILED DESCRIPTION

Systemic lupus erythematosus (SLE) T cells express high levels of calcium/calmodulin-dependent protein kinase type IV (CaMKIV), which translocates to the nucleus upon engagement of the T cell receptor (TCR)/CD3 and accounts for abnormal T cell function. We discovered that CaMKIV inhibition results in significant suppression of nephritis, autoantibody production, decreased expression of the costimulatory molecule CD86 on B cells, and suppression of IL-17 production, suggesting that overexpression of CaMKIV contributes to lupus pathology. Furthermore, we have discovered that CaMKIV inhibition results in reduced mesangial cell (MC) proliferation, where MC proliferation is implicated in the progression of various kidney disorders. Thus, inhibitors of CaMKIV may be useful in the treatment or amelioration of inflammatory autoimmune disorders, such as SLE, or kidney disorders, such as glomerulonephritis or lupus nephritis associated with SLE, in a subject that has been diagnosed with such a disorder or that is at risk of developing an inflammatory autoimmune disorder or a kidney disorder.

### Calcium/Calmodulin-Dependent Protein Kinase

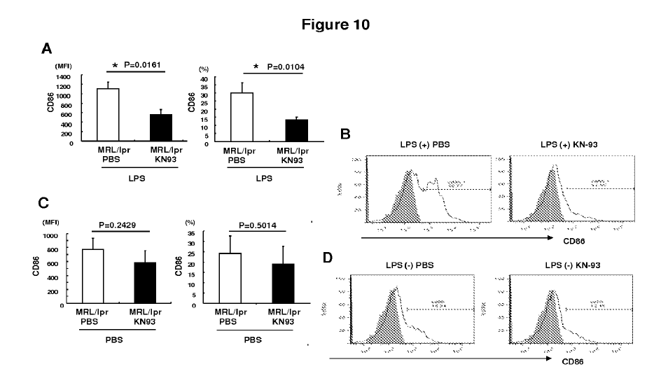

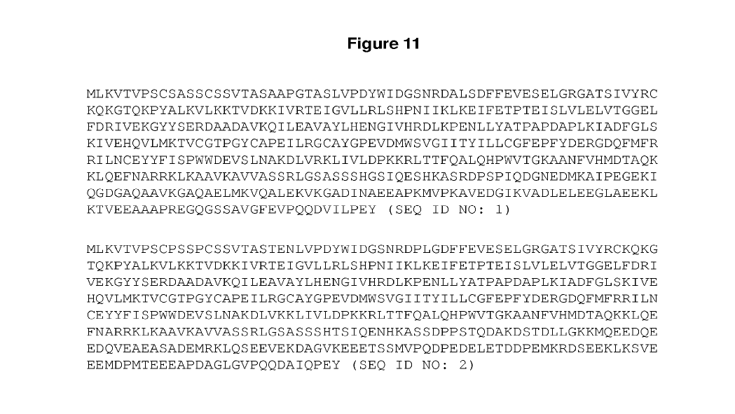

CaMKIV (SEQ ID NOs: 1-4 and FIGS. 11-13) is a serine/threonine-specific protein kinase that is primarily regulated by the calcium/calmodulin complex and is expressed primarily in the brain, T-lymphocytes, and post-meiotic germ cells. CaMKIV plays a role m the activity-dependent phosphorylation of CREB, which is required for CREB-mediated transcription.

The invention features inhibitor compounds that specifically inhibit or reduce the biological activity or expression of CaMKIV. Such inhibitor compounds can be used to treat or ameliorate inflammatory autoimmune disorders (e.g., SLE) or a kidney disorder (e.g., glomerulonephritis). CaMKIV inhibitor compounds can include any compound (peptidyl or non-peptidyl), small molecule, or nucleic acid (e.g., siRNA). In one example, the inhibitor compound is a small molecule inhibitor (e.g., KN-93). The CaMKIV inhibitor compound can also be a nucleic acid molecule that reduces or inhibits the expression of CaMKIV polypeptide or nucleic acid molecules, and exemplary siRNA molecules are provided in the Examples.

For any of the CaMKIV inhibitor compounds, a reduction in the biological activity of CaMKIV can be evaluated using any of the assays described herein including, but not limited to, assays for determining a reduction in CaMKIV protein expression levels or kinase assays. For example, a CaMKIV inhibitor compounds may result in decreased phosphorylation of CaMKIV at Thr200. Decreased phosphorylation of CaMKIV can be determined using, e.g., an ELISA screening assay in a high throughput system.

In one specific example, a CaMKIV inhibitor compound can be used to treat SLE or ameliorate the symptoms of SLE. In another specific example, a CaMKIV inhibitor compound can be used to treat glomerulonephritis or ameliorate the symptoms of glomerulonephritis.

### Inflammatory Autoimmune Disorders

The methods of the present invention may be used in the treatment or inhibition of inflammatory autoimmune disorders (e.g., SLE). Such methods may also be used to ameliorate symptoms of these disorders. Such disorders include, for example, Hashimoto's thyroiditis, pernicious anemia, Addison's disease, type I diabetes, rheumatoid arthritis, systemic lupus erythematosus (SLE), dermatomyositis, Sjögren's syndrome, lupus erythematosus (e.g., discoid lupus erythematosus, drug-induced lupus erythematosus, and neonatal lupus erythematosus), multiple sclerosis, myasthenia gravis, reactive arthritis, Grave's disease, and celiac disease (e.g., gluten sensitive enteropathy).

Additional disorders that may be treated using the methods of the present invention include, for example, juvenile onset diabetes mellitus, Wegener's granulomatosis, inflammatory bowel disease, polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyl, and telangiectasia), adult onset diabetes mellitus (e.g., type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

### Kidney Disorders

The methods of the present invention may be used in the treatment or inhibition of kidney disorders (e.g., glomerulonephritis) or such disorders associated with an inflammatory autoimmune disorder (e.g., lupus nephritis associated with SLE). In particular, the kidney disorder is associated with proliferation of mesangial cells. Exemplary kidney disorders include glomerulonephritis (e.g., membranoproliferative glomerulonephritis or post-streptococcal glomerulonephritis), IgA nephropathy, lupus nephritis, diabetic nephropathy, and glomerulosclerosis, or any other kidney disorder described herein.

### Therapeutic Compounds

Therapeutic compounds useful in the methods of the invention include any compound that can reduce or inhibit the biological activity or expression level of, e.g., CaMKIV.

Exemplary inhibitor compounds include, but are not limited to, peptidyl or non-peptidyl compounds that specifically bind CaMKIV; antisense nucleobase oligomers; morpholino oligonucleotides; small RNAs; small molecule inhibitors; compounds that decrease the half-life of the mRNA or protein of CaMKIV; compounds that decrease transcription or translation of CaMKIV; or compounds that reduce or inhibit the expression levels CaMKIV or decrease the biological activity of CaMKIV. Examples of nucleic acid and small molecule inhibitors are provided in the Examples below.

Inhibitor compounds of the present invention may reduce or inhibit the biological activity or expression levels of CaMKIV by at least 10%, 20%, 25%, 30%, 40%, 50%, 60%, 70%, 80%, 90%, 95%, 96%, 97%, 98%, 99%, or more. Preferably, the inhibitor compound can reduce or inhibit an inflammatory autoimmune disorder (e.g., SLE), a kidney disorder (e.g., glomerulonephritis), or symptoms of such disorders by at least 10%, 20%, 25%, 30%, 40%, 50%, 60%, 70%, 80%, 90%, 95%, 96%, 97%, 98%, 99%, or more.

Nucleic Acid Molecules

The present invention features inhibitory nucleic acid molecules that may be used for the treatment or amelioration of an inflammatory autoimmune disorder (e.g., SLE) or a kidney disorder (e.g., glomerulonephritis). Such inhibitory nucleic acid molecules are capable of, for example, mediating down-regulation of the expression of a CaMKIV polypeptide or nucleic acid encoding the same or mediating a decrease in the activity of CaMKIV. Examples of the inhibitory nucleic acids of the invention include, without limitation, antisense oligomers (e.g., morpholinos), double-stranded RNAs (dsRNAs) (e.g., small interfering RNAs (siRNAs) and short hairpin RNAs (shRNAs)), and aptamers.

Antisense Oligomers

The present invention features the use of antisense nucleobase oligomers to downregulate expression of mRNA encoding a polypeptide (e.g., CaMKIV). By binding to the complementary nucleic acid sequence (the sense or coding strand), antisense nucleobase oligomers are able to inhibit protein expression. For example, the antisense nucleobase oligomer may reduce CaMKIV polypeptide expression in a cell that expresses increased levels of CaMKIV by at least 10%, 20%, 30%, 40%, 50%, 60%, 70%, 80%, 90%, or greater relative to cells treated with a control oligonucleotide. Methods for selecting and preparing antisense nucleobase oligomers are well known in the art. Methods for assaying levels of protein expression are also well known in the art and include, for example, Western blotting, immunoprecipitation, and ELISA.

One example of an antisense nucleobase oligomer particularly useful in the methods and compositions of the invention is a morpholino oligomer. Morpholinos act by binding to a target sequence within an RNA and blocking molecules which might otherwise interact with the RNA. Therefore, morpholinos directed to a CaMKIV polypeptide that reduce or inhibit the expression levels or biological activity of CaMKIV are particularly useful in the methods of the invention that require the use of inhibitor compounds.

dsRNAs

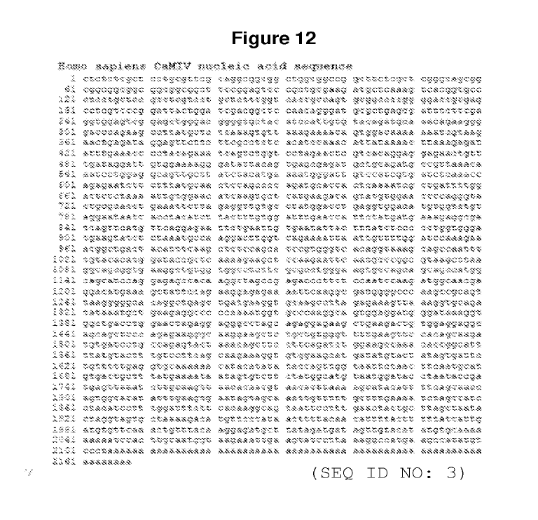

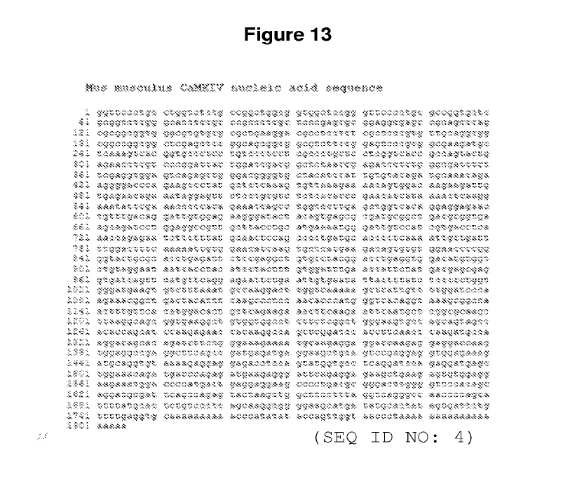

The present invention also features the use of double-stranded RNAs including, but not limited to, siRNAs and shRNAs. Short, double-stranded RNAs may be used to perform RNA interference (RNAi) to inhibit the expression of a polypeptide of the invention (e.g., CaMKIV). RNAi is a form of post-transcriptional gene silencing initiated by the introduction of dsRNA. Short (e.g., 15 to 32) nucleotide double-stranded RNAs, known generally as “siRNAs,” “small RNAs,” or “microRNAs,” are effective at down-regulating gene expression in nematodes (Zamore et al., Cell 101: 25-33) and in mammalian tissue culture cell lines (Elbashir et al., Nature 411:494-498, 2001). The further therapeutic effectiveness of this approach in mammals was demonstrated in vivo by McCaffrey et al. (Nature 418: 38-39, 2002). The small RNAs are at least 10 nucleotides, preferably 11, 12, 13, 14, 15, 16, 17, 18, 19, 20, 21, 22, 23, 24, 25, 26, 27, 28, 29, 30, 31, 32, 33, 34, 35 nucleotides in length and even up to 50 or 100 nucleotides in length (inclusive of all integers in between). Such small RNAs that are substantially identical to or complementary to any region of a polypeptide described herein are included in the invention. Non-limiting examples of small RNAs are substantially identical to (e.g., 60%, 65%, 70%, 75%, 80%, 85%, 90%, 95%, 96%, 97%, 98%, 99%, or 100% sequence identity) or complementary to the CaMKIV nucleic acid sequence (e.g., the human CaMKIV nucleic acid sequence (SEQ ID NO: 3, FIG. 12) or the murine CaMKIV nucleic acid sequence (SEQ ID NO: 4, FIG. 13). It should be noted that longer dsRNA fragments that are processed into small RNAs may be used. Small RNAs to be used as inhibitors of the invention can be identified by their ability to decrease polypeptide expression levels or biological activity performing assays known in the art or provided herein.

The specific requirements and modifications of small RNAs are known in the art and are described, for example, in PCT Publication No. WO 01/75164, and U.S. Patent Application Publication Nos. 2006/0134787, 2005/0153918, 2005/0058982, 2005/0037988, and 2004/0203145, the relevant portions of which are herein incorporated by reference.

siRNA molecules can be obtained and purified through a variety of protocols known to one of skill in the art, including chemical synthesis or recombinant production using a Drosophila in vitro system. They are commercially available from companies such as Dharmacon Research Inc. or Xeragon Inc., or they can be synthesized using commercially available kits such as the Silencer™ siRNA Construction Kit from Ambion or HiScribe™ RNAi Transcription Kit from New England BioLabs. Alternatively, siRNA can be prepared using standard procedures for in vitro transcription of RNA and dsRNA annealing procedures.

shRNAs can also be used in the methods of the invention. shRNAs are designed such that both the sense and antisense strands are included within a single RNA molecule and connected by a loop of nucleotides. shRNAs can be synthesized and purified using standard in vitro T7 transcription synthesis. shRNAs can also be subcloned into an expression vector, which can then be transfected into cells and used for in vivo expression of the shRNA.

A variety of methods are available for transfection of dsRNA into mammalian cells. For example, there are several commercially available transfection reagents useful for lipid-based transfection of siRNAs including, but not limited to, TransIT-TKO™ (Mirus), Transmessenger™ (Qiagen), Oligofectamine™ and Lipofectamine™ (Invitrogen), siPORT™ (Ambion), and DharmaFECT™ (Fisher Scientific). Agents are also commercially available for electroporation-based methods for transfection of siRNA, such as siPORTer™ (Ambion Inc.). Microinjection techniques may also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA, and such vectors are known in the art. Protocols for each transfection reagent are available from the manufacturer. Additional methods are known in the art and are described, for example, in U.S. Patent Application Publication No. 2006/0058255.

Aptamers

The present invention also features aptamers to the polypeptides of the invention (e.g., CaMKIV) and the use of such aptamers to down-regulate expression of the polypeptide or nucleic acid encoding the polypeptide. Aptamers are nucleic acid molecules that form tertiary structures that specifically bind to a target molecule. The generation and therapeutic use of aptamers are well established in the art. See, e.g., U.S. Pat. No. 5,475,096 and U.S. Patent Application Publication No. 2006/0148748. For example, a CaMKIV aptamer may be a pegylated, modified oligonucleotide, which adopts a three-dimensional conformation that enables it to bind to CaMKIV and inhibit the biological activity of CaMKIV.

Small Molecule Therapeutic Agents

Small molecule therapeutic agents for use in the present invention can be identified using standard screening methods specific to the target (e.g., CaMKIV). These screening methods can also be used to confirm the activities of derivatives of compounds found to have a desired activity, which are designed according to standard medicinal chemistry approaches. After a small molecule therapeutic agent is confirmed as being active with respect to a particular target, the therapeutic agent can be tested in vitro, as well as in appropriate animal model systems.

Examples of small molecule therapeutic agents (e.g., inhibitors of CaMKIV) include ST0609 (7-oxo-7H-benzimidazo[2,1-a]benz[de]isoquinoline-3-carboxylic acid acetate), KN-93 (N-[2-[[[3-(4-chlorophenyl)-2-propenyl]methylamino]methyl]phenyl]-N-(2-hydroxyethyl)-4-methoxybenzenesulphonamide), K252A, and derivatives, analogs, or mimetics thereof.

Therapeutic Administration and Formulation

The therapeutic agents described herein (e.g., inhibitors of CaMKIV) can be formulated and administered in a variety of ways (e.g., routes known for specific indications, including, but not limited to, topically, orally, subcutaneously, bronchial injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly). For example, a pharmaceutical composition containing an inhibitor of CaMKIV may be in the form of a pill, tablet, capsule, liquid, or sustained-release tablet for oral administration; a liquid for intravenous or subcutaneous administration; a polymer or other sustained-release vehicle for local administration; or an ointment, cream, gel, liquid, or patch for topical administration. Continuous systemic infusion or periodic injection of the therapeutic agent (e.g., inhibitor of CaMKIV) can also be used to treat, ameliorate, or reduce the likelihood of a disorder (e.g., an inflammatory autoimmune disorder (e.g., SLE) or a kidney disorder (e.g., glomerulonephritis)).

For parenteral administration, the therapeutic agents may be formulated in a unit dosage injectable form (e.g., solution, suspension, or emulsion) in association with a pharmaceutically acceptable parenteral vehicle. Such vehicles are inherently non-toxic and non-therapeutic. Examples of such vehicles include, e.g., water, saline, Ringer's solution, dextrose solution, liposomes, and 5% human serum albumin Nonaqueous vehicles, such as fixed oils and ethyl oleate, may also be used. The vehicle may contain minor amounts of additives, such as substances that enhance isotonicity and chemical stability (e.g., buffers and preservatives). Therapeutic agents typically are formulated in such vehicles at concentrations of about 1 mg/ml to 10 mg/ml.

Where sustained release administration of the therapeutic agent is desired in a formulation with release characteristics suitable for the treatment of, e.g., an inflammatory autoimmune disorder (e.g., SLE) or a kidney disorder (e.g., glomerulonephritis), microencapsulation of the therapeutic agent may be contemplated. See, e.g., Johnson et al., Nat Med. 2: 795-799, 1996; Yasuda, Biomed Ther. 27: 1221-1223, 1993; Hora et al., Bio/Technology 8: 755-758 1990; Cleland, “Design and Production of Single Immunization Vaccines Using Polylactide Polyglycolide Microsphere Systems,” in “Vaccine Design: The Subunit and Adjuvant Approach,” Powell and Newman, Eds., Plenum Press: New York, pp. 439-462, 1995; WO 97/03692; WO 96/40072; WO 96/07399; and U.S. Pat. No. 5,654,010, hereby incorporated by reference.

Sustained-release formulations may include those developed using poly-lactic-coglycolic acid (PLGA) polymer. The degradation products of PLGA, lactic and glycolic acids, can be cleared quickly from the human body. Moreover, the degradability of this polymer can be adjusted from months to years depending on its molecular weight and composition (see, e.g., Lewis, “Controlled release of bioactive agents from lactide/glycolide polymer,” in M. Chasin and Dr. Langer (Eds.), Biodegradable Polymers as Drug Delivery Systems (Marcel Dekker: New York, pp. 1-41, 1990).

Therapeutic formulations are prepared using standard methods known in the art by mixing the active ingredient having the desired degree of purity with optional physiologically acceptable carriers, excipients, or stabilizers in the form of lyophilized formulations or aqueous solutions (see, e.g., Remington's Pharmaceutical Sciences, 20^{th} edition, Ed. A. Gennaro, 2000, Lippincott, Williams & Wilkins, Philadelphia, Pa.). Acceptable carriers include, e.g., saline; buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagines, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

The therapeutic formulation may contain a pharmaceutically acceptable salt (e.g., sodium chloride), preferably at a physiological concentration. The formulations of the invention can also contain a pharmaceutically acceptable preservative. In some embodiments, the preservative concentration ranges from 0.1 to 2.0% v/v. Suitable preservatives include those known in the pharmaceutical arts (e.g., benzyl alcohol, phenol, m-cresol, methylparaben, and propylparaben). The formulations of the invention may also include a pharmaceutically acceptable surfactant (e.g., non-ionic detergents, Tween-20, or pluronic acid (F68)). Suitable surfactant concentrations are, e.g., 0.005 to 0.02%.

Administrations can be single or multiple (e.g., 2-, 3-, 6-, 8-, 10-, 20-, 50-, 100-, 150-, or more administrations). The composition can be administered at anytime (e.g., after diagnosis or detection of a disorder or a condition associated with the disorder (e.g., using the diagnostic methods known in the art or described herein) or before diagnosis of a disorder to a subject at risk of developing the disorder). Encapsulation of the therapeutic agent (e.g., inhibitor of CaMKIV) in a suitable delivery vehicle (e.g., polymeric microparticles or implantable devices) may increase the efficiency of delivery, particularly for oral delivery.

Administration of a therapeutic agent, alone or in combination with another therapeutic agent, can be one to four times daily for one day to one year, for example, 1 to 100 days, 1 to 60 days, or until the symptoms of the disorder are reduced or eliminated, and may even be for the life of the subject. Chronic, long-term administration may be required in some cases.

### Combination Therapies

The therapeutic agent(s) (e.g., an inhibitor of CaMKIV) of the present invention may be provided in conjunction (e.g., before, during, or after) with additional therapies to treat a disorder (e.g., an inflammatory autoimmune disorder (e.g., SLE) or a kidney disorder (e.g., glomerulonephritis)). Treatment therapies that can be used in combination with the methods of the invention include, but are not limited to, anti-inflammatory agents, analgesics, corticosteroids, immunosuppressants, and disease-modifying antirheumatic drugs.

Anti-Inflammatory Agents

Exemplary anti-inflammatory agents include non-steroidal anti-inflammatory drugs (e.g., ibuprofen, ketoprofen, piroxicam, indomethacin, diclofenac, sulindac, naproxen, aspirin, or tacrolimus), cyclooxygenase-2-specific inhibitors such as rofecoxib (Vioxx®) and celecoxib (Celebrex®), topical glucocorticoid agents, and specific cytokines directed at T lymphocyte function. Additional suitable anti-inflammatory agents include flubiprofen, diclofenac, and ketarolac.

Analgesics

Exemplary analgesics include dextropropoxyphene, co-codamol, hydrocodon, opiods (e.g., morphine, codeine, oxycodone, or methadone), fentanyl, procaine, lidocaine, tetracaine, dibucaine, benzocaine, p-buthylaminobenzoic acid 2-(diethylamino) ethyl ester HCl, mepivacaine, piperocaine, and dyclonine

Corticosteroids

Exemplary corticosteroids include corticosterone, cortisone, aldosterone, hydrocortisone (cortisol), hydrocortisone acetate, cortisone acetate, tixocortol pivalate, prednisolone, methylprednisolone, prednisone, triamcinolone acetonide, fludrocortisone, triamcinolone alcohol, mometasone, amcinonide, budesonide, desonide, fluocinonide, fluocinolone acetonide, halcinonide, betamethasone, betamethasone sodium phosphate, dexamethasone and dexamethasone derivatives, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

Immunosuppressants

Exemplary immunosuppressants include glucocorticoids, cyclophosphamide, nitrosoureas, platinum compounds, folic acid analogues (e.g., methotrexate), purine analogues (e.g., azathioprine or mercaptopurine), pyrimidine analogues, protein synthesis inhibitors, dactinomycin, anthracyclines, mitomycin C, bleomycin, mithramycin, muromonab, basiliximab, daclizumab, sirolimus, interferons, opiods, TNF-binding proteins (e.g., etanercept or adalimumab), curcumin, catechins, mycophenolic acid, fingolimod, or myriocin.

Disease-Modifying Antirheumatic Drugs

Exemplary disease-modifying antirheumatic drugs include chloroquine, hydroxychloroquine, ciclosporin (Cyclosporin A), D-penicillamine, etanercept, golimumab, gold salts (e.g., sodium aurothiomalate or auranofin), infliximab, leflunomide, methotrexate, minocycline, rituximab, and sulfasalazine.

In addition to the administration of therapeutic agents, the additional therapeutic regimen may involve, e.g., gene therapy, renal transplantation, or a modification to the lifestyle of the subject being treated. Such lifestyle changes may be helpful to control an inflammatory autoimmune disorder and include weight loss, physical exercise, diet control, reduction in alcohol intake, reduction in smoking, and avoidance of sunlight.

### Dosages

Generally, when administered to a human, the dosage of any of the therapeutic agents (e.g., inhibitors of CaMKIV) described herein may depend on the nature of the agent and can readily be determined by one skilled in the art. Typically, such dosage is about 0.001 mg to 2000 mg per day, about 1 mg to 1000 mg per day, or about 5 mg to 500 mg per day.

The dosage required depends on the choice of the route of administration; the nature of the formulation; the nature of the subject's disorder; the subject's size, weight, surface area, age, and sex; other drugs being administered; and the judgment of the subject's physician. Wide variations in the needed dosage are to be expected in view of the differing efficiencies of various routes of administration. For example, oral administration would be expected to require higher dosages than administration by intravenous injection. Variations in these dosage levels can be adjusted using standard empirical routines for optimization, as is well understood in the art. Additionally, pharmacogenomic information (e.g., the effect of genotype on the pharmacokinetic, pharmacodynamic, or efficacy profile of a therapeutic) about a particular subject may affect the dosage used.

### Diagnostic Methods

Alterations in the expression or biological activity of CaMKIV in a test sample, as compared to a normal reference, can be used to diagnose any of the inflammatory autoimmune disorders or kidney disorders of the invention.

A subject having an inflammatory autoimmune disorder, a kidney disorder, or a propensity to develop these disorders, may show an alteration (e.g., a decrease of 10%, 20%, 30%, 40%, 50%, 60%, 70%, 80%, 90%, or more) in the expression or biological activity of CaMKIV. In one example, a decrease in CaMKIV expression or biological activity in a subject sample, as compared to a normal reference, is indicative of an inflammatory autoimmune disorder or a risk of developing the same. CaMKIV can include full-length polypeptide, degradation products, alternatively spliced isoforms of the polypeptide, enzymatic cleavage products of the polypeptide, the polypeptide bound to a substrate or ligand, or free (unbound) forms of the polypeptide.

Standard methods may be used to measure polypeptide levels in any bodily fluid, including, but not limited to, urine, blood, serum, plasma, saliva, or cerebrospinal fluid. Such methods include immunoassay, ELISA, Western blotting using antibodies directed to a polypeptide of the invention (e.g., CaMKIV), and quantitative enzyme immunoassay techniques. In one example, an antibody that specifically binds CaMKIV is used in an immunoassay for the detection of CaMKIV and the diagnosis of any of the inflammatory autoimmune disorders (e.g., SLE) or kidney disorders (e.g., glomerulonephritis) described herein or the identification of a subject at risk of developing an inflammatory autoimmune disorder or a kidney disorder. The measurement of antibodies specific to a polypeptide of the invention (e.g., CaMKIV or fragment thereof) in a subject may also be used for the diagnosis of an inflammatory autoimmune disorder, a kidney disorder, or a propensity to develop these disorders.

Nucleic acid molecules encoding a polypeptide of the invention (e.g., CaMKIV), or fragments or oligonucleotides thereof that hybridize to a nucleic acid molecule encoding CaMKIV at high stringency, may be used as a probe to monitor expression of nucleic acid molecules encoding CaMKIV in the diagnostic methods of the invention. Any of the nucleic acid molecules above can also be used to identify subjects having a genetic variation, mutation, or polymorphism in a nucleic acid molecule that are indicative of a predisposition to develop an inflammatory autoimmune disorder (e.g., SLE) or a kidney disorder (e.g., glomerulonephritis). These polymorphisms may affect nucleic acid or polypeptide expression levels or biological activity. Detection of genetic variation, mutation, or polymorphism relative to a normal, reference sample can be used as a diagnostic indicator of a subject likely to develop an inflammatory autoimmune disorder, a kidney disorder, or a propensity to develop these disorders. Methods for detecting such alterations are standard in the art and are described in Sandri et al. (Cell 117: 399-412, 2004). In one example, Northern blotting or real-time PCR is used to detect mRNA levels (Bdolah et al., Am. J. Physio. Regul. Integre. Comp. Physiol. 292: R971-R976, 2007).

In one embodiment, hybridization at high stringency with PCR probes that are capable of detecting a CaMKIV nucleic acid molecule, including genomic sequences or closely related molecules, may be used to hybridize to a nucleic acid sequence derived from a subject having an inflammatory autoimmune disorder, a kidney disorder, or at risk of developing such disorders. The specificity of the probe, whether it is made from a highly specific region, e.g., the 5′ regulatory region, or from a less specific region, e.g., a conserved motif, and the stringency of the hybridization or amplification (e.g., maximal, high, intermediate, or low) determine whether the probe hybridizes to a naturally occurring sequence, allelic variants, or other related sequences. Hybridization techniques may be used to identify mutations in a nucleic acid molecule or may be used to monitor expression levels of a gene encoding, e.g., CaMKIV.

Diagnostic methods can include measurement of absolute levels of a polypeptide, nucleic acid, or antibody of the invention, or relative levels of a polypeptide, nucleic acid, or antibody of the invention as compared to a reference sample. In one example, an increase in the level or biological activity of a CaMKIV polypeptide, nucleic acid, or antibody, as compared to a normal reference, is considered a positive indicator of an inflammatory autoimmune disorder, a kidney disorder, or a propensity to develop these disorders.

In any of the diagnostic methods, the level of a polypeptide, nucleic acid, or antibody, or any combination thereof, can be measured at least two different times from the same subject and an alteration in the levels (e.g., by 10%, 20%, 30%, 40%, 50%, 60%, 70%, 80%, 90%, or more) over time is used as an indicator of an inflammatory autoimmune disorder, a kidney disorder, or the propensity to develop these disorders. It will be understood by the skilled artisan that for diagnostic methods that include comparing of the polypeptide, nucleic acid, or antibody level to a reference level, particularly a prior sample taken from the same subject, a change over time (e.g., an increase of CaMKIV) with respect to the baseline level can be used as a diagnostic indicator of an inflammatory autoimmune disorder (e.g., SLE), a kidney disorder (e.g., glomerulonephritis), or a predisposition to develop these disorders. The level of the polypeptide (e.g., CaMKIV), nucleic acid encoding the polypeptide, or antibody that binds the polypeptide in a bodily fluid sample of a subject having an inflammatory autoimmune disorder, a kidney disorder, or the propensity to develop such disorders may be altered, e.g., increased by as little as 10%, 20%, 30%, or 40%, or by as much as 50%, 60%, 70%, 80%, or 90% or more, relative to the level of the polypeptide, nucleic acid, or antibody in a prior sample or samples.

The diagnostic methods described herein can be used individually or in combination with any other diagnostic method described herein for a more accurate diagnosis of the presence of, severity of, or predisposition to an inflammatory autoimmune disorder or a kidney disorder.

### Subject Monitoring

The diagnostic methods described herein can also be used to monitor the progression of a disorder (e.g., an inflammatory autoimmune disorder (e.g., SLE) or a kidney disorder (e.g., glomerulonephritis)) during therapy or to determine the dosages of therapeutic compounds to be administered. In one embodiment, the levels of, for example, CaMKIV polypeptides are measured repeatedly as a method of diagnosing the disorder and monitoring the treatment or management of the disorder. In order to monitor the progression of the disorder in a subject, subject samples can be obtained at several time points and may then be compared. For example, the diagnostic methods can be used to monitor subjects during treatment with a therapeutic agent. In this example, serum samples from a subject can be obtained before treatment with a therapeutic agent, again during treatment with a therapeutic agent, and again after treatment with a therapeutic agent. In this example, the level of CaMKIV in a subject is closely monitored and, if the level of CaMKIV begins to increase during therapy, the therapeutic regimen for treatment of the disorder can be modified as determined by the clinician (e.g., the dosage of the therapy may be changed or a different therapeutic may be administered). The monitoring methods of the invention may also be used, for example, in assessing the efficacy of a particular drug or therapy in a subject, determining dosages, or in assessing progression, status, or stage of the disorder.

### Screening Assays

The methods of the present invention also include screening methods to identify compounds that modulate, alter, or decrease (e.g., by at least 10%, 20%, 30%, 40%, 50%, 60%, 70%, 80%, 90%, 95%, or more) the expression or biological activity of CaMKIV. Compounds that decrease the expression or biological activity of CaMKIV may be used for the treatment or amelioration of an inflammatory autoimmune disorder (e.g., SLE) or a kidney disorder (e.g., glomerulonephritis). Candidate compounds can be tested for their effect on CaMKIV biological activity (e.g., kinase activity) using assays known in the art.

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### Examples

The present invention is illustrated by the following examples, which are in no way intended to be limiting of the invention.

### Example 1

### Generation of CaMKIV-Deficient MRL/lpr Mice

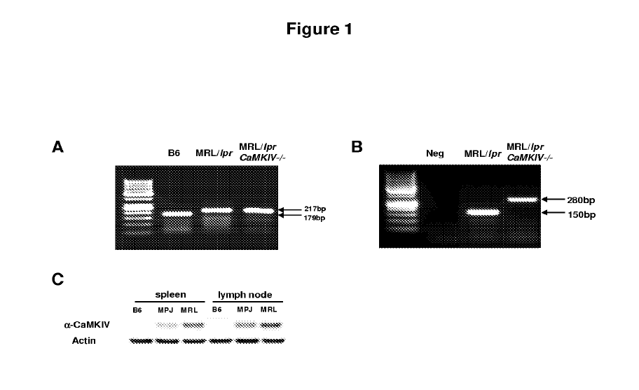

CaMKIV-deficient mice have been reported to display defects in positive selection in the thymus with a block in the generation of single positive T cells, but they do not manifest any obvious immune disease (Raman et al., J Immunol. 167: 6270-6278, 2001). CaMKIV-deficient mice have been derived by targeted disruption of exon III of the CaMKIV gene (Ho et al., J Neurosci. 20: 6459-6472, 2000). Here, MRL/lprCaMKIV−/− mice were generated (F6-F8) on a mixed MRL/lpr background and studied through the 6^{th} month of age. All mice described herein were purchased from The Jackson Laboratory and maintained in a SPF animal facility, and all experiments were approved by the Institutional Animal Care Committee of Beth Israel Deaconess Medical Center. Genotyping PCR for wild-type Fas (179 bp) and lpr mutation (217 bp) alleles in C57B6/L, MRL/lpr and MRL/lprCaMKIV−/− mice is shown (FIG. 1A). Wild-type (WT) (150 bp) and CaMKIV-null (280 bp) alleles were identified by PCR in MRL/lpr and MRL/lprCaMKIV−/− mice and are shown in FIG. 1B.

### Example 2

### CaMKIV is Overexpressed in Lymphoid Organs of MRL/Lpr Mice and CaMKIV Deficiency Suppresses Disease Expression in MRL/Lpr Mice

Spleen and lymph node were homogenized in radioimmunoprecipitation assay (RIPA) buffer at 4° C. After centrifugation at 14,000 rpm for 30 minutes at 4° C., supernatant was collected and stored at −80° C. until use. The following antibodies were used for the immunoblot assay: mouse anti-CaMKIV (BD Biosciences) and rabbit anti-actin (Sigma).

CaMKIV expression was significantly higher in spleen and lymph node extracts from MRL/lpr mice compared to C57BL/6 and MRL/MPJ mice (FIG. 1C). In addition, CaMKIV expression was higher in MRL/MPJ than in C57BL/6 mice.

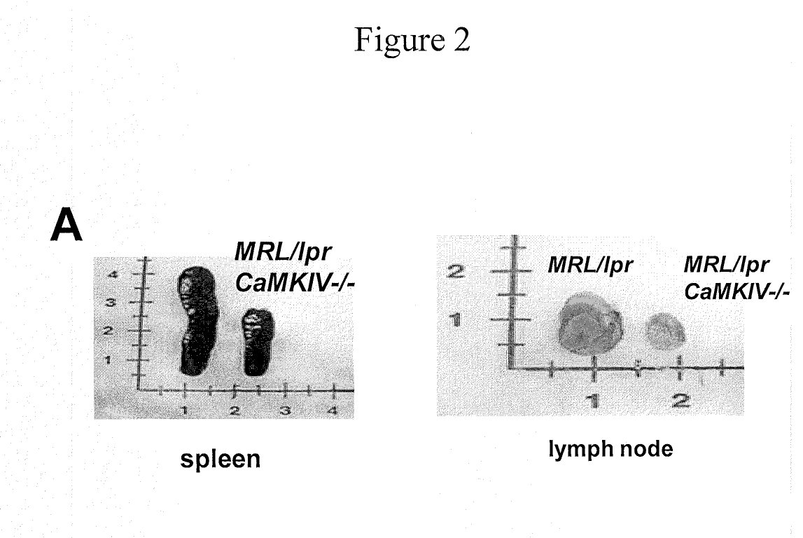

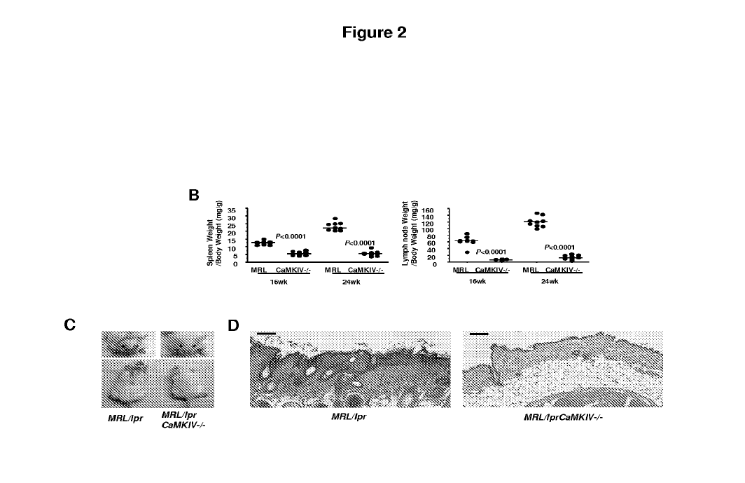

At 24 weeks of age, spleen and lymph node size (expressed as organ/body weight ratio) (FIGS. 2A and 2B) in the MRL/lprCaMKIV−/− mice compared to MRL/lpr mice were significantly reduced.

Other organs, including the kidneys, were smaller in the CaMKIV-deficient mice. We evaluated the kidney pathology and quantified the intensity of the disease in glomerular, tubule interstitial, and perivascular areas according to a previously described scoring system (Kikawada et al., J Immunol. 170: 3915-3925, 2003 and Sadanaga et al., Arthritis Rheum. 56: 1618-1628, 2007). In addition, the extent of skin injury (FIG. 2C), as evaluated by thickness of the epidermis and infiltration of the dermis by inflammatory cells (FIG. 2D), was notably decreased in the MRL/lprCaMKIV−/− mice compared to MRL/lpr mice. To prepare the skin sections, the skin was removed, fixed in 10% buffered formalin, and embedded in paraffin. Sections (5-μm) were stained with Hematoxylin-Eosin (HE) for light microscopic observation.

### Example 3

### CaMKIV Deficiency Improves Lupus Nephritis

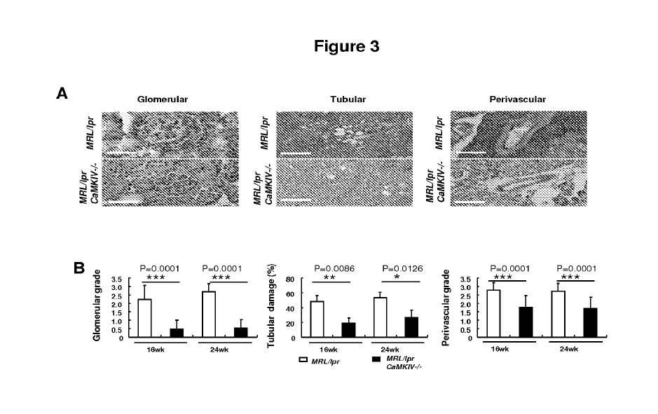

The severity of nephritis was evaluated in a blinded manner by histological examination of the kidney sections. The kidneys of the mice were removed, fixed in 10% buffered formalin, and embedded in paraffin. Sections (5-μm) were stained with Hematoxylin-Eosin (HE) for light microscopic observation. We evaluated separately glomerular, tubular and perivascular areas, and the presence of glomerular crescents in order to obtain accurate measurements of the disease using a previously described scoring system (Kikawada et al., J Immunol. 170: 3915-3925, 2003 and Sadanaga et al., Arthritis Rheum. 56: 1618-1628, 2007). Glomerular lesions and tubular and perivascular infiltrates were significantly decreased in MRL/lprCaMKIV−/− compared to MRL/lpr mice. Representative sections are shown in FIG. 3A, and cumulative data are shown in FIG. 3B. In FIG. 3B, all values are expressed as mean+SD. A Kruskal-Wallis test with post-hoc comparisons using the Scheffe's test were employed for inter-group comparisons of multiple variables. Statistical analyses were performed using StatView software (Abacus Concepts). A level of P≦0.05 was considered statistically significant.

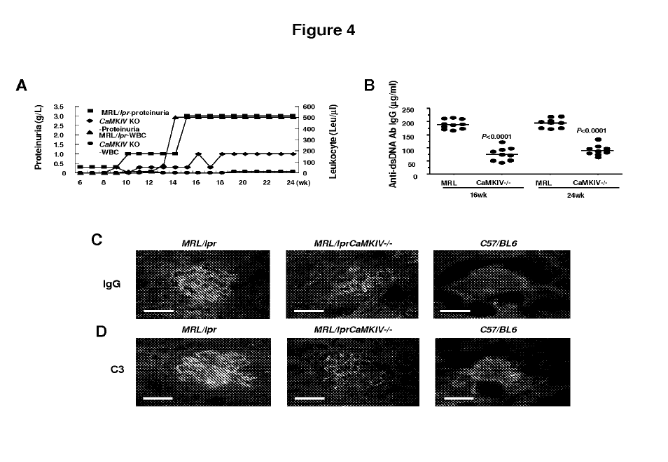

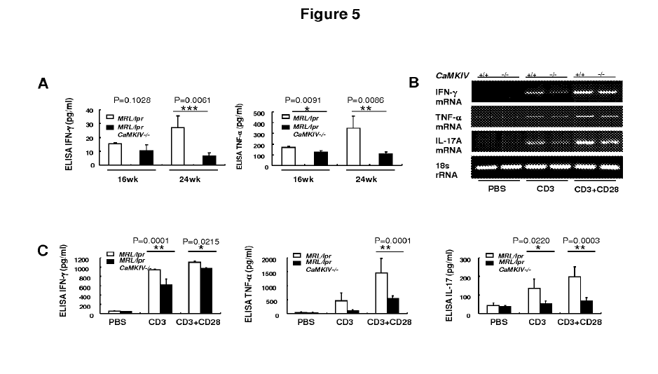

We recorded proteinuria and pyuria in CaMKIV-deficient and sufficient MRL/lpr mice from the 6^{th} to the 24^{th} week of age. The mice in each group were placed overnight in a Nalgene metabolic cage to collect urine. Urine was measured with Multistix 10SG reagent strips and analyzed by a Clinitek Status analyzer (Bayer Healthcare). Proteinuria increased to 3 g/L in MRL/lpr mice at the age of 14 weeks and remained at the same level thereafter. However, in MRL/lprCaMKIV−/− mice, proteinuria remained at 1 g/L throughout the observation period. The number of leukocytes in the urine increased to 500 μl at week 14 in MRL/lpr mice, whereas it remained less than 15 μl in MRL/lprCaMKIV−/− mice (FIG. 4A).

Typical of MRL/lpr mice is the development of anti-dsDNA antibodies (Theofilopoulos et al., Adv Immunol. 37: 269-390, 1985). Serum anti-dsDNA antibodies were detected using a mouse anti-dsDNA IgG ELISA kit (Alpha Diagnostic). The levels of IgG anti-dsDNA antibody were significantly decreased in MRL/lprCaMKIV−/− groups as compared to MRL/lpr mice at weeks 16 and 24 weeks of age (FIG. 4B).

C3 and IgG are deposited in the kidneys of lupus prone mice and patients with lupus nephritis. These deposits are found in the mesangial and pericapillary regions and are considered to contribute to the inflammatory process. To determine the amount of IgG and C3 deposited in the kidneys of MRL/lprCaMKIV−/− mice, immunofluorescence assays were performed Immunofluorescence was performed using frozen sections (4-μm) fixed in cold (−20° C.) acetone for 10 minutes and then air dried. Sections were incubated with primary antibodies at room temperature for 1 hour. Subsequently, sections were washed three times in PBS and incubated with secondary antibodies at room temperature for 30 minutes. After three washes with PBS, Fluoromount-G (Southern Biotech) was applied and sections were scanned in a Nikon Eclipse Ti confocal microscope. Images were analyzed with EZ-Cl v.3.7 software.

The amounts of IgG (FIG. 4C) and C3 (FIG. 4D) found deposited in the kidneys of MRL/lprCaMKIV−/− mice were significantly less compared to MRL/lpr mice and comparable to those noticed in the kidneys of control healthy C57BL/6 mice. These data demonstrate that CaMKIV deficiency protects lupus-prone MRL/lpr mice from renal disease.

### Example 4

### CaMKIV Deficiency Suppresses Pro-Inflammatory Cytokine Production in MRL/lpr Mice

IFN-γ (Baccala et al., Immunol Rev. 204: 9-26, 2005) and TNF-α(Jacob et al., J Autoimmunol. 5(A): 133-143, 1992) contribute to the expression of autoimmunity and lupus nephritis in lupus-prone mice. IL-17-producing cells are increased in SLE patients and have been claimed to contribute to the expression of lupus pathology in humans (Crispin et al., J Immunol. 181: 8761-8766, 2008) and lupus-prone mice (Kang et al., J Immunol. 178: 7849-7858, 2007 and Odegard et al., J Exp Med. 205: 2873-2886, 2008).

Accordingly, we determined the expression of these cytokines in CaMKIV-sufficient and deficient MRL/lpr mice. First, we measured IFN-γ, TNF-α and IL-17A expression in serum using an ELISA kit (R&D Systems). Serum IFN-γ levels were decreased 75% and TNF-α was decreased 69% in MRL/lprCaMKIV−/− mice compared to MRL/lpr mice at 24 weeks of age (FIG. 5A). IL-17A was undetectable in the sera.

Next, we isolated splenocytes from MRL/lpr and MRL/lprCaMKV−/− mice at the age of 24 weeks. Two million splenocytes were incubated in 1 ml of RPMI 1640 supplemented with 10% FCS and stimulated with phosphate buffered saline (PBS), anti-CD3, or anti-CD3/CD28 antibodies for 72 hours. At the end of the culture period, supernatants were collected and RNA was extracted from the cells using a RNeasy Mini Kit (Qiagen). cDNA was produced using random primers from an equal amount of RNA. The following primers were designed using Primer3 software (Rozen et al., Methods Mol Biol. 132: 365-386, 2000):

IL-17A Forward:
(SEQ ID NO: 5)
5′-CAGCAGCGATCATCCCTCAAAG-3′
IL-17A Reverse:
(SEQ ID NO: 6)
5′-CAGGACCAGGATCTCTTGCTG-3′
TNF-α Forward:
(SEQ ID NO: 7)
5′-GGCAGGTCTACTTTGGAGTCATTGC-3′
TNF-α Reverse:
(SEQ ID NO: 8)
5′-ACATTCGAGGCTCCAGTGAATTCGG-3′
IFN-γ Forward:
(SEQ ID NO: 9)
5′-CACGGCACAGTCATTGAAAGCC-3′
IFN-γ Reverse:
(SEQ ID NO: 10)
5′-CTTATTGGGACAATCTCTTCCC-3′
Human IL-17A Forward:
(SEQ ID NO: 11)
5′-CGAAATCCAGGATGCCC-3′
IL-17A Reverse:
(SEQ ID NO: 12)
5′-GACACCAGTATCTTCTCCA G-3′
18srRNA Forward:
(SEQ ID NO: 13)
5′-ACTCAACACGGGAAACCTCA-3′
18srRNA Reverse:
(SEQ ID NO: 14)
5′-AACCA GACAAATCGCTCCAC-3′

TNF-α, IFN-γ, and IL-17A expression were significantly decreased after stimulation with anti-CD3 or anti-CD3 and anti-CD28 antibodies in MRL/lprCaMKIV−/− mice compared to MRL/lpr mice (FIGS. 5B and 5C). Similar to the reported experiments using ex vivo T cells from patients with SLE (Juang et al., J Clin Invest. 115: 996-1005, 2005), spleen cells from MRL/lprCaMKIV−/− mice spontaneously produced more IL-2 than MRL/lpr mice (data not shown). Expression levels of IL-4 and IL-10 by spleen cells were increased in MRL/lprCaMKIV−/− mice compared to MRL/lpr mice (data not shown).

### Example 5

### CaMKIV Deficiency Results in Suppression of IL-17A Production

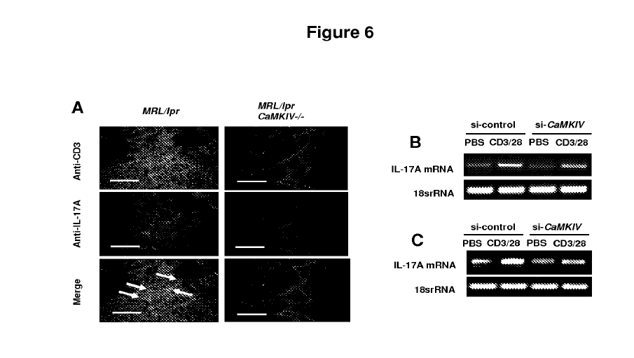

Because we have found IL17-producing T cells in the kidneys of patients with lupus nephritis (Crispin et al., J Immunol. 181: 8761-8766, 2008), we stained kidney sections with anti-CD3 (eBioscience) and anti-IL-17A antibodies (BD Pharmigen). As shown in the immunofluorescence experiments of FIG. 6A, kidney sections from MRL/lpr mice contained a significant number of CD3⁺IL-17A⁺ cells in the tubulointerstitial area, whereas no such cells were found in the sections from kidneys form MRL/lprCaMKIV−/− mice.

Because of the profound effect of CaMKIV inhibition on the expression of IL-17A production and IL-17⁺CD3⁺ infiltration of kidney tissues in MRL/lpr mice (FIG. 6A) and the parallel of these findings to human SLE, we wished to determine whether silencing of CaMKIV in T cells from patients with SLE would result in suppression of IL-17 production. Human SLE T cells were obtained from the peripheral blood of patients with SLE or matched controls as described previously (Sunahori et al., J Immunol. 182: 1500-1508, 2009) under an Institutional Review Board-approved protocol and transfected with CaMKIV siRNA or control siRNA as described previously (Juang et al., J Clin Invest. 115: 996-1005, 2005).

For siRNA transfection, T cells were purified from peripheral blood of patients with SLE and age- and sex-matched controls using Rosettesep (Stemcell Technologies). T cells were electroporated in the presence of CaMKIV siRNA (Qiagen) or control siRNA using an Amaxa nucleofector (Lonza). After 48 hours of rest, cells were stimulated with PBS or CD3/CD28. After 5 hours, cells were lysed for RNA extraction. RNA was extracted by homogenizing T cells and isolating total RNA using a RNeasy Mini Kit (Qiagen).

Normal and SLE T cells transduced with CaMKIV siRNA for 48 hours, but not with scrambled control siRNA, suppressed IL-17A mRNA (FIGS. 6B and 6C), and transduction of SLE T cells with a CaMKIV vector promoted IL-17 expression (data not shown). Production of IL-17A in the culture supernatant followed the same pattern (data not shown).

### Example 6

### Treatment of MRL/lpr Mice with the CaMKIV Inhibitor KN-93 Ameliorates Lupus Nephritis

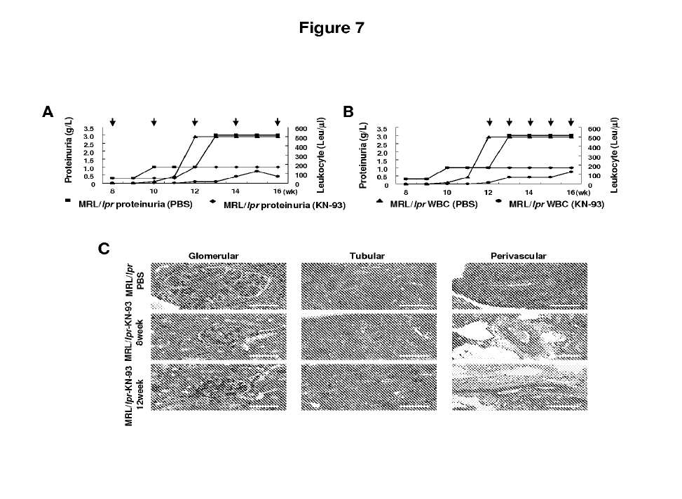

Although genetic deficiency of CaMKIV effectively prevented the development of lupus nephritis, it may have done so through unrecognized developmental mechanisms. To rule out such possibility, we used KN-93, a well-known CaMKIV inhibitor (Tsung et al., J Exp Med. 204: 2913-2923, 2007; Sato et al., Nat Med. 12: 1410-1416, 2006; and Ilario et al., Blood 111: 723-731, 2008), to treat MRL/lpr mice and determine its effect on the expression of lupus nephritis. The agent was administered by intraperitoneal injections at a dose of 0.08 mg/mouse, three times a week. We treated MRL/lpr mice with PBS (control) or KN-93 either every other week from week 8 to week 16 (disease prevention) (FIG. 7A) or weekly from week 12 to week 16 (disease treatment) (FIG. 7B).

In the disease prevention experiment, KN-93 administration was started either before the onset of proteinuria, when the mice were 8 weeks old. These mice received the agent every other week. In the disease treatment experiment, the effectiveness of KN-93 in established disease was evaluated. KN-93 administration was started when mice were 12 weeks old and continued three times a week during 5 weeks. Mice of both experiments were sacrificed at the end of their 16th week of age.

Both treatment schemes produced the same beneficial effect on lupus nephritis. Whereas the PBS-treated mice developed proteinuria (3 g/L) and pyuria (500 leukocytes/μl), KN-93-treated mice had less than 1 g/L protein and fewer than 125 leukocytes/μl in the urine. KN-93 treated mice had less kidney pathology, as determined histopathologically. Kidneys of treated mice did not have any glomerular crescents and the glomerular damage, interstitial, and perivascular inflammatory cell infiltration were limited (FIG. 7C).

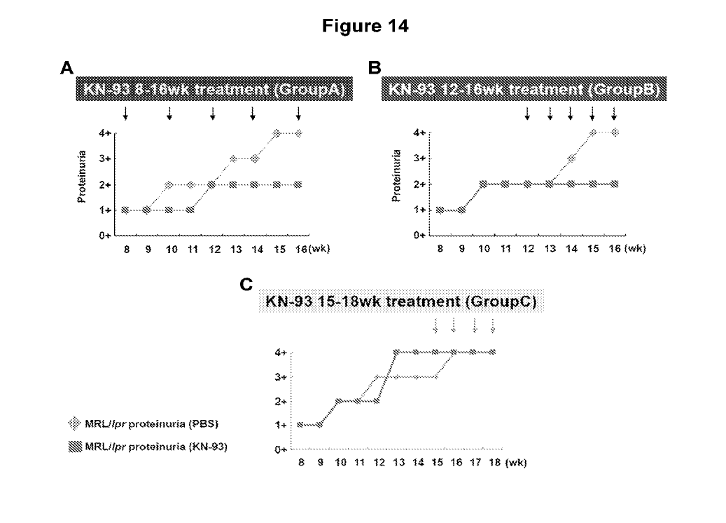

Additional experiments are provided in FIGS. 14A-14B, where these results were comparable to those in FIGS. 7A-7B. In contrast, FIG. 14C provides a different treatment scheme, where mice were treated with PBS (control) or KN-93 weekly from week 15 to week 18.

The fact that treatment of MRL/lpr mice with KN-93 prior to and after the initiation of the disease suggests that CaMKIV contributes to the expression of autoimmunity and organ damage through mechanisms unrelated to thymic selection.

### Example 7

### Decreased Expression of CD86, but not CD80, in Spleen B Cells of MRL/lprCaMKIV −/− mice

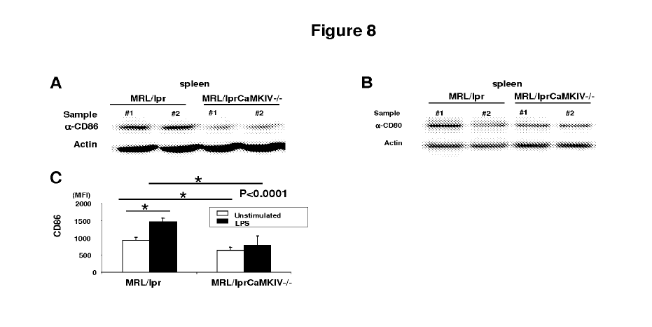

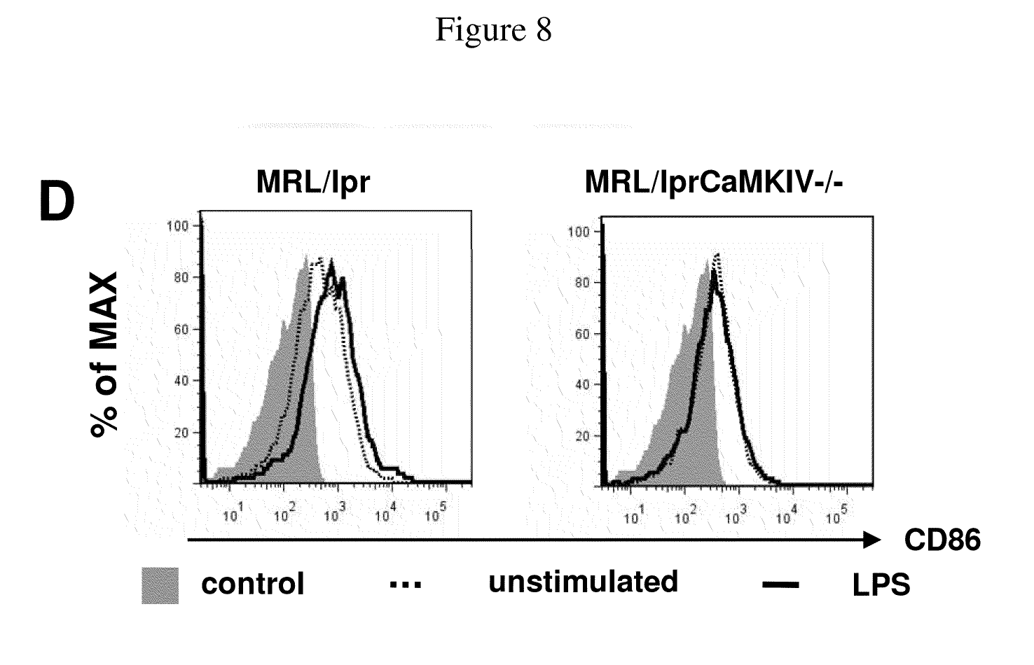

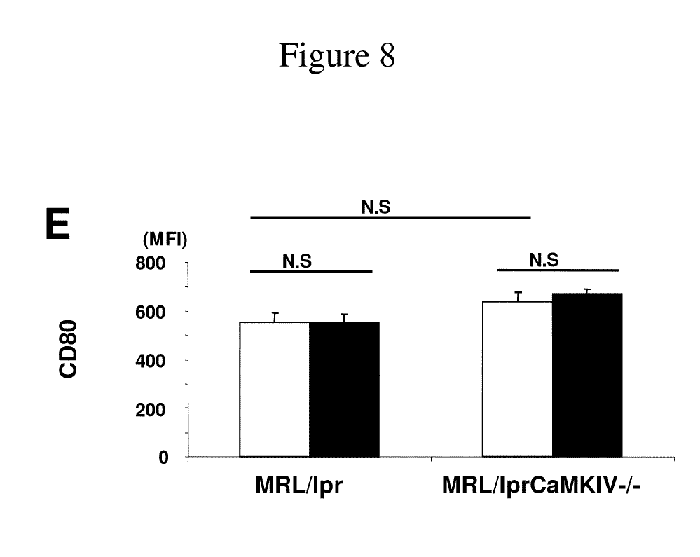

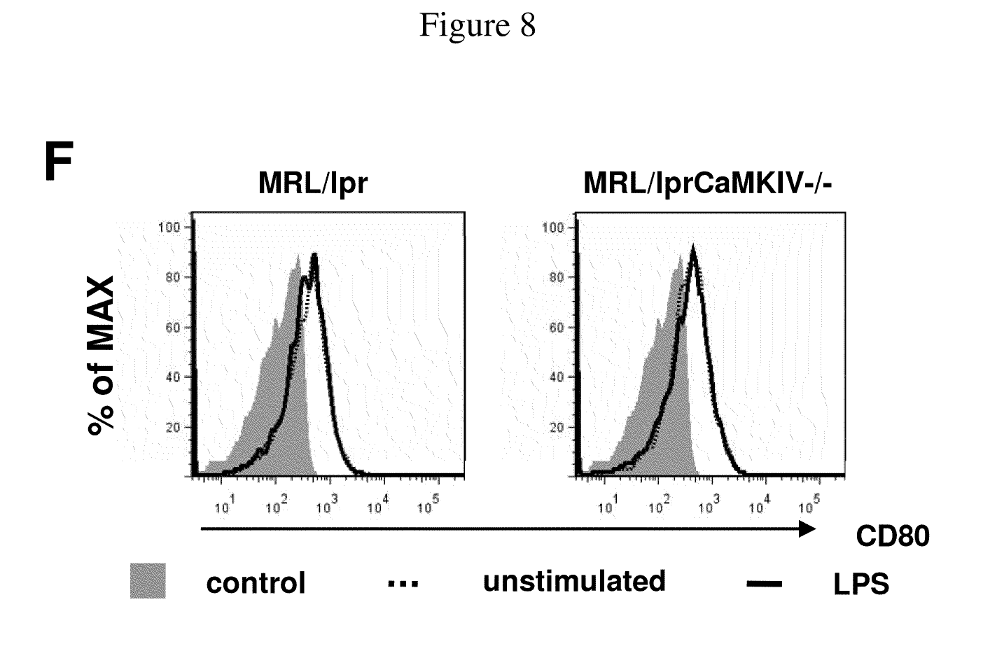

B7 costimulatory molecules (CD80 and CD86) provide signals essential for T cell activation. The B7-CD28 interactions promote T cell growth, survival, and differentiation (Salomon et al., Annu Rev Immunol. 19:225-252, 2001), and CD86 has been shown previously to be necessary for the development of organ damage in the MRL/lpr mouse (Liang et al., J Immunol. 165: 3436-3443, 2000). We studied the expression of the B7 (CD80 and CD86) costimulatory molecules in CaMKIV-deficient mice. Protein extracts were obtained from the spleens of 24-week old MRL/lpr and MRL/lprCaMKIV−/− mice and immunoblotted for CD86 (FIG. 8A) and CD80 (FIG. 8B) using rabbit anti-CD86 (Santa Cruz Biotechnology) and rabbit anti-CD80 (Abcam) antibodies, respectively. The expression of CD86, but not CD80, was significantly decreased in MRL/lprCaMKIV−/− compared with MRL/lpr spleen.

Next, we obtained spleen cell suspensions from MRL/lpr and MRL/lprCaMKIV−/− mice, stimulated them with LPS (1 μg/ml) or PBS for 24 hours, and stained them for CD86 surface expression. The mean fluorescence intensity (MFI) of CD86, but not of CD80, was significantly increased in MRL/lpr compared with MRL/lprCaMKIV−/− mice, especially in stimulated cells (FIGS. 8C, 8D, 8E, and 8F), as determined via flow cytometry.

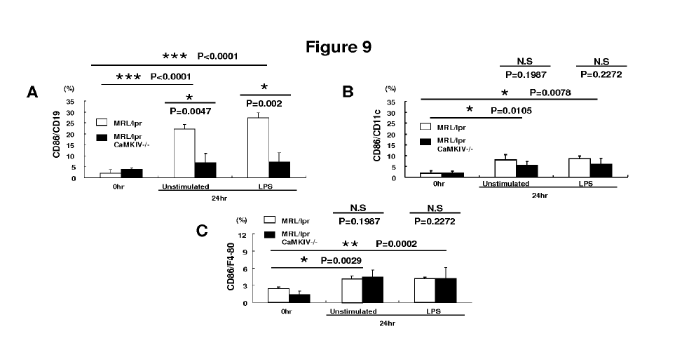

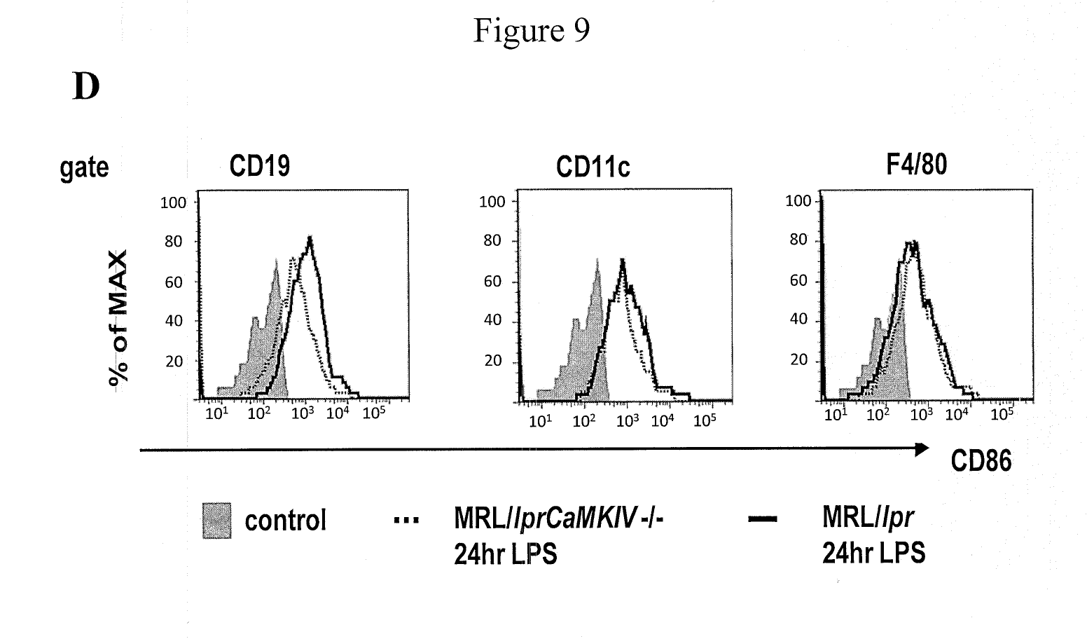

We wished to determine whether CaMKIV deficiency suppresses CD86 expression in all types of antigen presenting cells (APC). Splenocytes were isolated from MRL/lpr and MRL/lprCaMKIV−/− mice and stimulated with LPS (1 μg/ml) or PBS for 24 hours. We stained cells with fluorochrome-conjugated antibodies against the major histocompatibility complex (MHC) class II (eBioscience) (all APCs), CD19 (B cells), CD11c (dendritic cells), and F4/80 (macrophages) (BioLegend). Samples were placed in a LSRII flow cytometer (BD Biosciences), and analysis was performed with FlowJo v. 7.5.3 (Tree Star). Thirty-thousand T cells were acquired for analysis. CD86 expression was noted significantly decreased in MRL/lprCaMKIV−/− mice among MHCII positive cells (data not shown). Similarly, CD86 expression was found significantly decreased in CD19 positive B cells in MRL/lprCaMKIV−/− compared with MRL/lpr, especially in stimulated cells (FIGS. 9A and 9D). However, we did not observe a clear difference among CaMKIV-sufficient and deficient MRL/lpr mice in CD11c positive dendritic cells (FIGS. 9B and 9D) and F4/80 positive macrophages (FIGS. 9C and 9D).

Splenocytes isolated from MRL/lpr mice were stimulated with LPS (1 μg/ml) or PBS for 24 hours in the presence or absence of KN-93. CD19 positive B cells were analyzed for the expression of CD86. We noted that the presence of KN-93 suppressed the expression of CD86 on stimulated cells (FIGS. 10A and 10B). A trend towards lower expression of CD86 was noted in non-LPS stimulated cells if KN-93 was present (FIGS. 10C and 10D).

In conclusion, we have provided evidence that increased IL-17 and IFN′ production and increased expression of CD86 represent pathways through which increased CaMKIV may lead to autoimmunity and relevant pathology. In T cells, increased expression of CaMKIV leads to increased expression of IL-17, and IL-17 producing cells enter target organs, such as the kidney. In B cells, increased expression of CaMKIV leads to increased expression of CD86, which may account for the increased production of autoantibody, and facilitates interaction with T cells. Additional immune pathways may also be involved. It remains possible that increased expression of CaMKIV in non-immune tissues may engage additional non-immune mechanisms.

We provide herein in vivo and in vitro evidence that KN-93 may be further developed for the treatment of patients with SLE. We show that KN-93 not only prevents, but also suppresses, established disease in MRL/lpr mice. The agreement between human and murine data further strengthens this claim. Since CaMKIV is also expressed in neuronal cells and is important for their proper function, timing and dosing will determine the therapeutic efficacy of CaMKIV inhibitors.

### Example 8

### KN-93 Decreases Cytokine Production in Normal Human T Cells and Macrophages

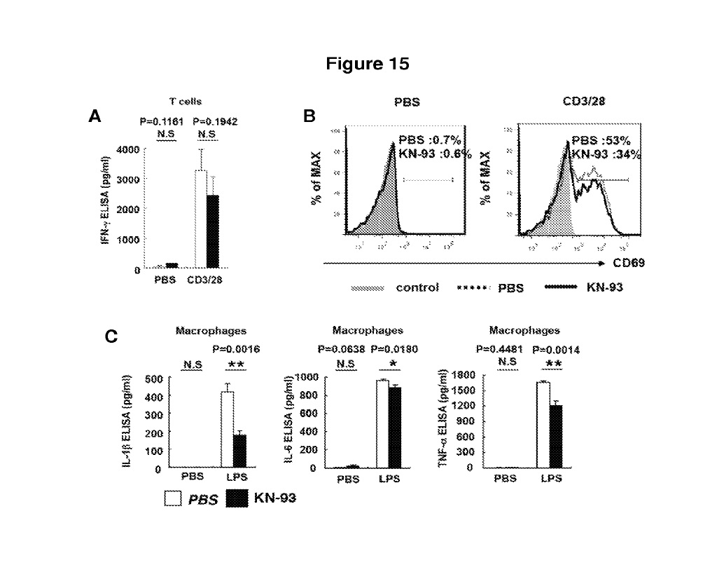

Normal human T cells and macrophages were treated with PBS (control) or KN-93. Overall, treatment with KN-93 decreased expression of CD69 on T cells (FIG. 15B) and decreased production of various cytokines in T cells and macrophages, including IFN-γ, IL-1β, IL-6, and TNF-α(FIGS. 15A and 15C). Furthermore, the activity of IL-6 in murine mesangial cells decreases in the absence of CaMKIV. Taken together, inhibition of CaMKIV by KN-93 or absence of CaMKIV (e.g., by genetic deficiency) decreases the expression of various pro-inflammatory cytokines, and inhibitors of CaMKIV could be used to treat disorders associated with one or more of such cytokines (e.g., autoimmune disorders).

### Example 9

### Inhibition of CaMKIV with KN-93 Suppresses Mesangial Cell Proliferation

Mesangial cell proliferation is associated with numerous disorders, including SLE and glomerulonephritis. The following experiments were conducted to assess the effect of inhibiting CaMKIV on mesangial cell proliferation.

Briefly, four hundred thousand primary mesangial cells were incubated in 2 mL of RPMI 1640 with 10% FBS. Cells were plated in 6-well plates and made quiescent by serum starvation for 24 hours. Then, cells were treated with 20 ng/ml of PDGF-BB (PeproTech, Inc. Rocky Hill, N.J.) for 24 hours. In some experiments, cells were pretreated with KN-93 (20 μM) for 48 hours before the addition of PDGF-BB.

For cell cycle analysis, mesangial cells were harvested with trypsin, washed twice with PBS, fixed in cold 95% ethanol, and stored at 4° C. until use. Before flow cytometric analysis, cells were washed with PBS and centrifuged, and the cell pellets were resuspended in a solution of RNAse (0.5 mg/ml) in PBS and incubate at 37° C. for 20 minutes. Then, propidium iodide (PI) (40 μg/ml) was added in PBS for 30 minutes. Stained cells were analyzed with a flow cytometer (model FACS Scan; BD Bioscience, Franklin Lakes, N.J.). Data were acquired using CellQuest software (BD Bioscience); at least 10,000 events were collected for each histogram. Analysis was performed with FlowJo v. 7.6.1 (Tree Star).

For Western blot analysis, mesangial cells were homogenized in radioimmunoprecipitation assay (RIPA) buffer at 4° C. After centrifugation at 14,000 rpm for 30 min at 4° C., supernatant was collected and stored at −80° C. until use. The following antibodies were used for immunoblot assay: rabbit anti-CDK2, rabbit anti-cyclin D1 (Cell signaling) and rabbit anti-actin (Sigma).

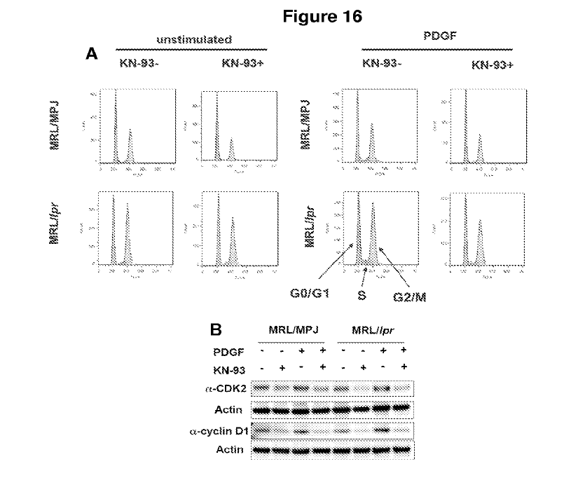

These experiments show that inhibition of CaMKIV with KN-93 suppressed mesangial cell proliferation with or without PDGF stimulation, where PDGF enhances further mesangial cell proliferation (FIG. 16A). Furthermore, K93-inhibition of CaMKIV decreased the presence of kinases involved in cell cycle proliferation, such as CD-K2 and cyclin D1 (FIG. 16B). Taken together, an inhibitor of CaMKIV (e.g., KN-93 or any described herein) could be used to reduce mesangial cell (MC) proliferation and to treat diseases associated with MC proliferation (e.g., any disorder described herein).

### Example 10

### Genetic Elimination of CaMKIV Suppressed Mesangial Cell Proliferation

In view of our results in Example 9, further experiments were conducted to assess mesangial cell proliferation in cells lacking CaMKIV. Briefly, isolated mesangial cells were grown in RPMI 1640 with 10% FBS. Four hundred thousand primary mesangial cells were plated 6-well plates and made quiescent by serum starvation for 24 hours. Then, cells were treated with 20 ng/mL of PDGF-BB (PeproTech, Inc. Rocky Hill, N.J.) for 24 hours. Cell cycle and Western blot analyses were conducted as described above in Example 9.

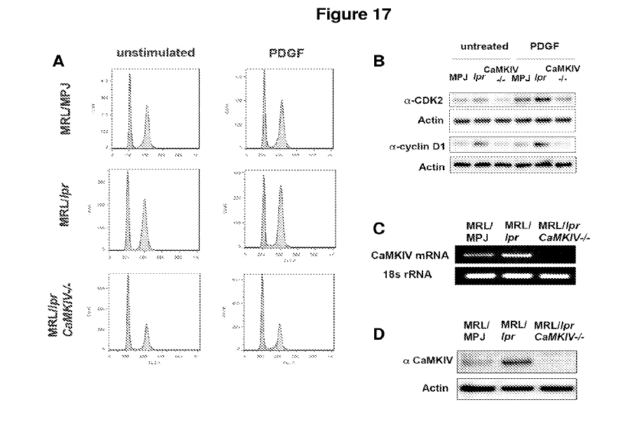

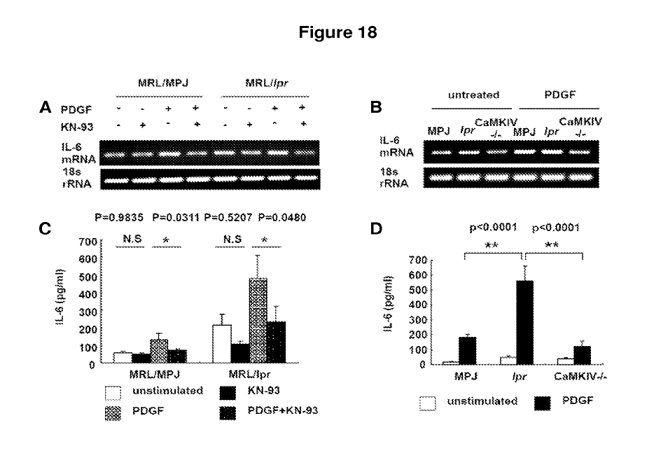

These experiments were conducted with cells from MRL/MPJ, MRL/lpr, and MRL/lprCaMKIV−/− mice, where levels of CaMKIV mRNA and protein are provided in FIGS. 17C-17D. Overall, these experiments show that genetic elimination of CaMKIV suppressed mesangial cell proliferation (FIG. 17A) and decreased the presence of kinases involved in cell cycle proliferation, such as CD-K2 and cyclin D1 (FIG. 17B).

### OTHER EMBODIMENTS

From the foregoing description, it is apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

What is claimed is:

1. A method of treating or reducing the likelihood of an inflammatory autoimmune disorder or a kidney disorder in a subject, said method comprising providing to said subject an inhibitor of calcium/calmodulin-dependent protein kinase type IV (CaMKIV), wherein said inhibitor is provided in an amount and for a duration that together are sufficient to treat or reduce the likelihood of said inflammatory autoimmune disorder or said kidney disorder in said subject.

2. The method of claim 1, wherein said inhibitor reduces or inhibits the biological activity or expression level of a CaMKIV protein or nucleic acid molecule.

3. The method of claim 2, wherein said biological activity of CaMKIV is kinase activity.

4. The method of claim 1, wherein said inhibitor of CaMKIV is a small molecule.

5. The method of claim 4, wherein said small molecule is KN-93.

6. The method of claim 1, wherein said inhibitor of CaMKIV is a nucleic acid.

7. The method of claim 6, wherein said nucleic acid is siRNA.

8. The method of claim 1, wherein said method further comprises providing an additional therapeutic agent to said subject.

9. The method of claim 8, wherein said additional therapeutic agent is adalimumab, azathioprine, chloroquine, hydroxychloroquine, ciclosporin, D-penicillamine, etanercept, golimumab, auranofin, infliximab, leflunomide, methotrexate, minocycline, rituximab, sulfasalazine, plaquenil, cyclophosphamide, tacrolimus, sirolimus, dehydroepiandrosterone, an opiate, an interferon, a corticosteroid, or a nonsteroidal anti-inflammatory drug.

10. A method of diagnosing a subject as having an inflammatory autoimmune disorder or a kidney disorder, said method comprising determining the level or biological activity of a CaMKIV nucleic acid or polypeptide, or fragments thereof, in a sample from said subject and comparing it to a reference, wherein an increase in the level or biological activity of said CaMKIV nucleic acid or polypeptide, or fragments thereof, compared to said reference is a diagnostic indicator of an inflammatory autoimmune disorder or a kidney disorder in said subject.

11. The method of claim 10, wherein said sample is a bodily fluid, cell, or tissue sample from said subject in which said CaMKIV nucleic acid or polypeptide is normally detectable.

12. The method of claim 11, wherein said bodily fluid is selected from the group consisting of urine, blood, serum, plasma, and cerebrospinal fluid.

13. A method of identifying a candidate compound useful for treating an inflammatory autoimmune disorder or a kidney disorder in a subject, said method comprising:
(a) contacting a CaMKIV polypeptide, or a fragment thereof, with a compound; and
(b) measuring the biological activity of said CaMKIV polypeptide, or fragment thereof, wherein a decrease in CaMKIV biological activity in the presence of said compound relative to CaMKIV biological activity in the absence of said compound identifies said compound as a candidate compound for treating an inflammatory autoimmune disorder or a kidney disorder in a subject.

14. The method of claim 13, wherein said biological activity is kinase activity.

15. A method of identifying a candidate compound useful for treating an inflammatory autoimmune disorder or a kidney disorder in a subject, said method comprising:
(a) contacting a cell or cell extract comprising a polynucleotide encoding CaMKIV with a compound; and
(b) measuring the level of CaMKIV expression in said cell or cell extract, wherein a decreased level of CaMKIV expression in the presence of said compound relative to the level in the absence of said compound identifies said compound as a candidate compound for treating an inflammatory autoimmune disorder or a kidney disorder in a subject.

16. The method of claim 1, wherein said inflammatory autoimmune disorder is Hashimoto's thyroiditis, pernicious anemia, Addison's disease, type I diabetes, rheumatoid arthritis, systemic lupus erythematosus (SLE), dermatomyositis, Sjögren's syndrome, lupus erythematosus, multiple sclerosis, myasthenia gravis, reactive arthritis, Grave's disease, or celiac disease.

17. The method of claim 16, wherein said inflammatory autoimmune disorder is SLE.

18. The method of claim 1, wherein said inflammatory autoimmune disorder is associated with a kidney disorder.

19. The method of claim 18, wherein said inflammatory immune disorder is SLE associated with lupus nephritis or glomerulonephritis.

20. The method claim 1, wherein said kidney disorder is glomerulonephritis, IgA nephropathy, lupus nephritis, diabetic nephropathy, or glomerulosclerosis.

21. The method of claim 20, wherein said kidney disorder is glomerulonephritis.
